SEQUENCE LISTING (71 sequences)

SEQ ID NO: 1 - DNA, 7286 bp, Homo sapiens
aggttatgtaagggtttgctttcaccccattcaaaaggtacctcttcctcttctcttgctccctctcgccctcattcttgtgcctatgcagacatttgagtagaggcgaatcactttcacttctgctggggaaattgcaacacgcttctttaaatggcagagagaaggagaaaacttagatcttctgataccaaatcactggaccttagaaggtcagaaatctttcaagccctgcaggaccgtaaaatgcgcatgtgtccaacggaagcactggggcatgagtggggaaggaatagaaacagaaagagggtaagagaagaaaaaagggaaagtggtgaaggcagggaggaaaattgcttagtgtgaatatgcacgcattcatttagttttcaaatccttgttgagcatgataaaattcccagcatcagacctcacatgttggtttccattaggatctgcctgggggaatatctgctgaatcagtggctctgagctgaactaggaaattcaccataattaggagagtcactgtatttctctccaaaaaaaaaaaagttatacccgagagacaggatcttctgatctgaaattttcttcacttctgaaattctctggtttgtgctcatcgttggtagctatttgttcatcaagagttgtgtagctggcttcttctgaaaaaaggaatctgcgtcatatctaagtcagatttcattctggtgctctcagagcagttagcccaggaaaggggccagcttctgtgacgactgctgcagaggcaggtgcagtttgtgtgccacagatattaactttgataagcacttaatgagtgccttctctgtgcgagaatggggaggaacaaaatgcagctcctaccctcctcgggctttagttgtaccttaataacaggaattttcatctgcctggctcctttcctcaaagaacaaagaagactttgcttcattaaagtgtctgagaaggaagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 2 - protein, 777 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 3 - DNA, 19 bp, Artificial Sequence
  Primer
ccagcagtgtgcttgctca

SEQ ID NO: 4 - DNA, 20 bp, Artificial Sequence
  Primer
cagactccaagcagcgaaga

SEQ ID NO: 5 - DNA, 23 bp, Artificial Sequence
  Primer
ccagaggtactcacaccatgaac

SEQ ID NO: 6 - DNA, 19 bp, Artificial Sequence
  Primer
ccagggaagttcagagtcc

SEQ ID NO: 7 - DNA, 21 bp, Artificial Sequence
  Primer
gccaccgttggtgccagtctg

SEQ ID NO: 8 - DNA, 23 bp, Artificial Sequence
  Primer
gtcaaaggtgctttggtctgtgg

SEQ ID NO: 9 - DNA, 20 bp, Artificial Sequence
  Primer
ccagcatgagaccagatgta

SEQ ID NO: 10 - DNA, 20 bp, Artificial Sequence
  Primer
aagcttcatcagagcacacc

SEQ ID NO: 11 - DNA, 22 bp, Artificial Sequence
  Primer
cttccactgctcttttgaagaa

SEQ ID NO: 12 - DNA, 24 bp, Artificial Sequence
  Primer
gacagcacaattacctatgtgctg

SEQ ID NO: 13 - DNA, 24 bp, Artificial Sequence
  Primer
cagcacaattacctatgtgctgga

SEQ ID NO: 14 - DNA, 24 bp, Artificial Sequence
  Primer
cttccaggttcattccagcctgaa

SEQ ID NO: 15 - DNA, 24 bp, Artificial Sequence
  Primer
ccaggttcattccagcctgaagac

SEQ ID NO: 16 - DNA, 23 bp, Artificial Sequence
  Primer
ggaattcagcaggccactacagg

SEQ ID NO: 17 - DNA, 20 bp, Artificial Sequence
  Primer
caggccactacaggagtctc

SEQ ID NO: 18 - DNA, 23 bp, Artificial Sequence
  Primer
ctggtattgcctttgcccatttc

SEQ ID NO: 19 - DNA, 24 bp, Artificial Sequence
  Primer
gtttcaggaacttacacctggatg

SEQ ID NO: 20 - DNA, 16 bp, Artificial Sequence
  Primer
cacagcaggtttgcac

SEQ ID NO: 21 - DNA, 23 bp, Artificial Sequence
  Primer
tcattaataatcagatcaggagc

SEQ ID NO: 22 - DNA, 23 bp, Artificial Sequence
  Primer
gcagagaatgactctaccctgca

SEQ ID NO: 23 - DNA, 19 bp, Artificial Sequence
  Primer
cctgcatgtacgaccaatg

SEQ ID NO: 24 - DNA, 16 bp, Artificial Sequence
  Primer
gagagaagcagtaagg

SEQ ID NO: 25 - DNA, 20 bp, Artificial Sequence
  Primer
cctgaagagagaagcagtaa

SEQ ID NO: 26 - DNA, 23 bp, Artificial Sequence
  Primer
tcctaaggacggtctgaagagcc

SEQ ID NO: 27 - DNA, 23 bp, Artificial Sequence
  Primer
aaccgctgccagttctggctgga

SEQ ID NO: 28 - DNA, 21 bp, Artificial Sequence
  Primer
ttcatgcatagaatccaagag

SEQ ID NO: 29 - DNA, 16 bp, Artificial Sequence
  Primer
tacgcactacatgtgg

SEQ ID NO: 30 - DNA, 20 bp, Artificial Sequence
  Primer
tggcaacagaagcagttgag

SEQ ID NO: 31 - DNA, 21 bp, Artificial Sequence
  Primer
cagctgtttaagatgggcagc

SEQ ID NO: 32 - DNA, 24 bp, Artificial Sequence
  Primer
ccagataaccagctgtaacacagc

SEQ ID NO: 33 - DNA, 20 bp, Artificial Sequence
  Primer
cacattcccatctgtcacca

SEQ ID NO: 34 - DNA, 21 bp, Artificial Sequence
  Primer
ccactgaccaatttggaagcc

SEQ ID NO: 35 - DNA, 19 bp, Artificial Sequence
  Primer
tgggtcagagcctcagcaa

SEQ ID NO: 36 - DNA, 19 bp, Artificial Sequence
  Primer
ggagggctgtatgtgaaag

SEQ ID NO: 37 - DNA, 16 bp, Artificial Sequence
  Primer
ccacatgtagtgcgta

SEQ ID NO: 38 - DNA, 18 bp, Artificial Sequence
  Primer
catcccaacaatcttggc

SEQ ID NO: 39 - DNA, 22 bp, Artificial Sequence
  Primer
gctcatcgacaactataggagg

SEQ ID NO: 40 - DNA, 22 bp, Artificial Sequence
  Primer
gtgcagaatctcataggttgcc

SEQ ID NO: 41 - DNA, 16 bp, Artificial Sequence
  Primer
aaacaggagtcactgg

SEQ ID NO: 42 - DNA, 16 bp, Artificial Sequence
  Primer
ctgccaattcggtaca

SEQ ID NO: 43 - DNA, 22 bp, Artificial Sequence
  Primer
cctcctatagttgtcgatgagc

SEQ ID NO: 44 - DNA, 20 bp, Artificial Sequence
  Primer
atattcactgatggactcca

SEQ ID NO: 45 - DNA, 19 bp, Artificial Sequence
  Primer
tcactgatggactccaaag

SEQ ID NO: 46 - DNA, 30 bp, Artificial Sequence
  Primer
ttcactgatggactccaaagaatcattaac

SEQ ID NO: 47 - DNA, 30 bp, Artificial Sequence
  Primer
tgatattcactgatggactccaaagaatca

SEQ ID NO: 48 - DNA, 18 bp, Artificial Sequence
  Primer
gagcggctcctctgccag

SEQ ID NO: 49 - DNA, 18 bp, Artificial Sequence
  Primer
gctcctctgccagagttg

SEQ ID NO: 50 - DNA, 16 bp, Artificial Sequence
  Primer
gaactgcggacggtgg

SEQ ID NO: 51 - DNA, 16 bp, Artificial Sequence
  Primer
tgcggcgggaactgcg

SEQ ID NO: 52 - DNA, 25 bp, Artificial Sequence
  Primer
ttaaggcagtcacttttgatgaaac

SEQ ID NO: 53 - DNA, 21 bp, Artificial Sequence
  Primer
ccattcttattaaggcagtca

SEQ ID NO: 54 - DNA, 30 bp, Artificial Sequence
  Primer
ttattaaggcagtcacttttgatgaaacag

SEQ ID NO: 55 - DNA, 30 bp, Artificial Sequence
  Primer
aggcaaccattcttattaaggcagtcactt

SEQ ID NO: 56 - DNA, 19 bp, Artificial Sequence
  Primer
cctctacaggacaaactga

SEQ ID NO: 57 - DNA, 16 bp, Artificial Sequence
  Primer
caacaaaacctctaca

SEQ ID NO: 58 - DNA, 21 bp, Artificial Sequence
  Primer
ccaagcagcagaggattctcc

SEQ ID NO: 59 - DNA, 23 bp, Artificial Sequence
  Primer
cagcagcaccgcagccagattta

SEQ ID NO: 60 - DNA, 23 bp, Artificial Sequence
  Primer
gtcaaaggtgctttggtctgtgg

SEQ ID NO: 61 - DNA, 21 bp, Artificial Sequence
  Primer
ccaaggactctcattcgtctc

SEQ ID NO: 62 - DNA, 2379 bp, Mus musculus
atggactccaaagaatccttagctccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcgcagcagcagcagcagcagcagcagcagcagcagcagcagcagcagcagcagccgcagccagatttatccaaagccgtttcactgtccatgggactgtatatgggagagaccgaaacaaaagtgatggggaatgacttgggctacccacagcagggccagcttggcctctcctctggggaaacagactttcggcttctggaagaaagcattgcaaacctcaataggtcgaccagccgtccagagaaccccaagagttcaacacctgcagctgggtgtgctaccccgacagagaaggagtttccccagactcactctgatccatcttcagaacagcaaaatagaaaaagccagcctggcaccaacggtggcagtgtgaaattgtataccacagaccaaagcacctttgacatcttgcaggatttggagttttctgccgggtccccaggtaaagagacaaacgagagtccttggaggtcagacctgttgatagatgaaaacttgctttctcctttggcgggagaagatgatccattccttctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatataattgggaataaaatgtctgccatttctgttcatggcgtgagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaagccctggaatgagaccagatgtgagttctcctccgtccagctcctccacagcaacgggaccacctcccaaactctgcctggtgtgctccgatgaagcttcgggatgccattatggggtgctgacgtgtggaagctgtaaagtcttctttaaaagagcagtggaaggacagcacaattacctttgtgctggaagaaatgattgcatcattgataaaattcgaagaaaaaactgtccagcatgccgctatcgaaaatgtcttcaagctggaatgaacctggaagctcgaaaaacgaagaaaaaaattaaaggaattcagcaagccactgcaggagtctcacaagacacttctgaaaacgctaacaaaacaatagttcctgccgcgctgccacagcttacccctaccctggtgtcactgctggaggtgatcgagcctgaggtgttatatgcaggatatgacagctctgttccagactcagcatggagaattatgaccacgctcaacatgttaggtgggcgccaagtgattgccgcagtgaaatgggcaaaggcgataccaggattcagaaacttacacctggatgaccaaatgacccttctacagtactcatggatgtttctcatggcatttgccctgggttggagatcatacagacaagcaagtggaaacctgctatgctttgctcctgatctgattattaatgagcagagaatgactctaccctgcatgtatgaccaatgtaaacacatgctgtttatctccactgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccattgtcaaaagggaaggaaactccagtcagaattggcagcggttttatcaactgacaaaacttttggactccatgcatgatgtggttgaaaatctccttagctactgcttccaaacatttttggataagtccatgagtattgaattcccagagatgttagctgaaatcatcactaatcagataccaaaatactcaaatggaaatatcaaaaagcttctgtttcatcagaaatga

SEQ ID NO: 63 - DNA, 1399 bp, Homo sapiens
atggactccaaagaatcattaactccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcgcagcagcagcagcagcagcagcagcagcagcagcagcagcagcagcagcagccgcagccagatttatccaaagccatttcactgtccatgggactgtatatgggagagaccgaaacaaaagtgatggggaatgacttgggctacccacagcagggccagcttggcctctcctctggggaaacagactttcggcttctggaagaaagcattgcaaacctcaataggtcgaccagccgtccagagaaccccaagagttcaacacctgcagctgggtgtgctaccccgacagagaaggagtttccccagactcactctgatccatcttcagaacagcaaaatagaaaaagccagcctggcaccaacggtggcagtgtgaaattgtatgccacagaccaaagcaccttagacatcttgcaggatttggagttttctgccgggtccccaggtaaagagacaaacgagagtccttggaggtcagacctgttgatagatgaaaacttgctttctcctttggcgggagaagatgatccattccttctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatatgattgggaataaaatgtctgccatttctgttcatggcgtgagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaggccctggaatgagaccagatgtgagttctcctccgtccagctcctccacagcaacgggaccacctcccaaactctgcctggtgtgctccgatgaagcttcgggatgccattatggggtgctgacgtgtggaagctgtaaagtcttcttcaaaagagcagtggaag

SEQ ID NO: 64 - DNA, 1333 bp, Homo sapiens
atggactccaaagaatcattaactccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcgcagcagcagcagcagcagcagcagcagcagcagcagcagcagcagcagcagccgcagccagatttatccaaagccatttcactgtccatgggactgtatatgggagagaccgaaacaaaagtgatggggaatgacttgggctacccacagcagggccagcttggcctctcctctggggaaacagactttcggcttctggaagaaagcattgcaaacctcaataggtcgaccagccgtccagagaaccccaagagttcaacacctgcagctgggtgtgctaccccgacagagaaggagtttccccagactcactctgatccatcttcagaacagcaaaatagaaaaagccagcctggcaccaacggtggcagtgtgaaattgtataccacagaccaaagcaccttagacatcttgcaggatttggagttttctgccgggtccccaggtaaagagacaaacgagagtccttggaggtcagacctgttgatagatgaaaacttgctttctcctttggcgggagaagatgatccattccctctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatataattgggaataaaatgtctgccatttctgttcatggcgtgagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaagccctggaatgagaccagatgtgagttctcctccgtccagctcctccacagcaacgggaccacctcccaaactctgcctggtgtgctctgatgaagctt

SEQ ID NO: 65 - DNA, 1332 bp, Homo sapiens
tggactccaaagaatcattaactccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcgcagcagcagcagcagcagcagcagcagcagcagcagcagcagcagcagcagccgcagccagatttatccaaagccatttcactgtccatgggactgtatatgggagagaccgaaacaaaagtgatggggaatgacttgggctacccacagcagggccagcttggcctctcctctggggaaacagactttcggcgtctggaagaaagcattgcaaacctcaataggtcgaccagccgtccagagaaccccaagagttcaacacctgcagctgggtgtgctaccccgacagagaaggagtttccccagactcactctgatccatcttcagaacagcaaaatagaaaaagccagcctggcaccaacggtggcagtgtgaaattgtataccacagaccaaagcaccttagacatcttgcaggatttggagttttctgccgggtccccaggtaaagagacaaacgagagtccttggaggtcagacctgttgatagatgaaaacttgctttctcctttggcgggagaagatgatccattccttctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatataattgggaataaaatgtctgccatttctgttcatggcgttagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaagccctggagtgagaccagatgtgagttctcctccgtccagctcctccacagcaacgggaccacctcccaaactctgcctggtgtgctctgatgaagctt

SEQ ID NO: 66 - DNA, 1333 bp, Homo sapiens
atggactccaaagaatcattaactccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaacgcgcagcagcagcagcagcagcagcagcagcagcagcagcagcagcagcagcagccgcagccagatttatccaaagccatttcactgtccatgggactgtatatgggagagaccgaaacaaaagtgatggggaatgacttgggctacccacagcagggccagcttggcctctcctctggggaaacagactttcggcttctggaagaaagcattgcaaacctcaataggtcgaccagccgtccagagaaccccaagagttcaacacctgcagctgggtgtgctaccccgacagagaaggagtttccccagactcactctgatccatcttcagaacagcaaaatagaaaaagccagcctggcaccaacggtggcagtgtgaaattgtataccacagaccaaagcaccttagacatcttgcaggatttggagttttctgccgggtccccaggtaaagagacaaacgagagtccttggaggtcagacctgttgatagatgaaaacttgctttctcctttggcgggagaagatgatccattccttctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatataattgggaataaaatgtctgccatttctgttcatggcgtgagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaagccctggaatgagaccagatgtgagttctcctccgtccagctcctccacagcaacgggaccacctcccaaactctgcctggtgtgctctgatgaagctt

SEQ ID NO: 67 - protein, 792 aa, Mus musculus
MDSKESLAPPGRDEVPSSLLGRGRGSVMDLYKTLRGGATVKVSASSPSVAAASQADSKQQRILLDFSKGSASNAQQQQQQQQQQQQQQQQQPQPDLSKAVSLSMGLYMGETETKVMGNDLGYPQQGQLGLSSGETDFRLLEESIANLNRSTSRPENPKSSTPAAGCATPTEKEFPQTHSDPSSEQQNRKSQPGTNGGSVKLYTTDQSTFDILQDLEFSAGSPGKETNESPWRSDLLIDENLLSPLAGEDDPFLLEGDVNEDCKPLILPDTKPKIQDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDNLTSLGAMNFAGRSVFSNGYSSPGMRPDVSSPPSSSSTATGPPPKLCLVCSDEASGCHYGVLTCGSCKVFFKRAVEGQHNYLCAGRNDCIIDKIRRKNCPACRYRKCLQAGMNLEARKTKKKIKGIQQATAGVSQDTSENANKTIVPAALPQLTPTLVSLLEVIEPEVLYAGYDSSVPDSAWRIMTTLNMLGGRQVIAAVKWAKAIPGFRNLHLDDQMTLLQYSWMFLMAFALGWRSYRQASGNLLCFAPDLIINEQRMTLPCMYDQCKHMLFISTELQRLQVSYEEYLCMKTLLLLSSVPKEGLKSQELFDEIRMTYIKELGKAIVKREGNSSQNWQRFYQLTKLLDSMHDVVENLLSYCFQTFLDKSMSIEFPEMLAEIITNQIPKYSNGNIKKLLFHQK

SEQ ID NO: 68 - protein, 466 aa, Homo sapiens
MDSKESLTPPGRDEVPSSLLGRGRGSVMDLYKTLRGGATVKVSASSPSVAAASQADSKQQRILLDFSKGSASNAQQQQQQQQQQQQQQQQQPQPDLSKAISLSMGLYMGETETKVMGNDLGYPQQGQLGLSSGETDFRLLEESIANLNRSTSRPENPKSSTPAAGCATPTEKEFPQTHSDPSSEQQNRKSQPGTNGGSVKLYATDQSTLDILQDLEFSAGSPGKETNESPWRSDLLIDENLLSPLAGEDDPFLLEGDVNEDCKPLILPDTKPKIQDTGDTILSSPSSVALPQVKTEKDDFIELCTPGVIKQEKLGPVYCQASFSGTNMIGNKMSAISVHGVSTSGGQMYHYDMNTASLSQQQDQKPVFNVIPPIPVGSENWNRCQGSGEDNLTSLGAMNFAGRSVFSNGYSGPGMRPDVSSPPSSSSTATGPPPKLCLVCSDEASGCHYGVLTCGSCKVFFKRAVE

SEQ ID NO: 69 - protein, 444 aa, Homo sapiens
MDSKESLTPPGRDEVPSSLLGRGRGSVMDLYKTLRGGATVKVSASSPSVAAASQADSKQQRILLDFSKGSASNAQQQQQQQQQQQQQQQQQPQPDLSKAISLSMGLYMGETETKVMGNDLGYPQQGQLGLSSGETDFRLLEESIANLNRSTSRPENPKSSTPAAGCATPTEKEFPQTHSDPSSEQQNRKSQPGTNGGSVKLYTTDQSTLDILQDLEFSAGSPGKETNESPWRSDLLIDENLLSPLAGEDDPFPLEGDVNEDCKPLILPDTKPKIQDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDNLTSLGAMNFAGRSVFSNGYSSPGMRPDVSSPPSSSSTATGPPPKLCLVCSDEA

SEQ ID NO: 70 - protein, 443 aa, Homo sapiens
DSKESLTPPGRDEVPSSLLGRGRGSVMDLYKTLRGGATVKVSASSPSVAAASQADSKQQRILLDFSKGSASNAQQQQQQQQQQQQQQQQQPQPDLSKAISLSMGLYMGETETKVMGNDLGYPQQGQLGLSSGETDFRRLEESIANLNRSTSRPENPKSSTPAAGCATPTEKEFPQTHSDPSSEQQNRKSQPGTNGGSVKLYTTDQSTLDILQDLEFSAGSPGKETNESPWRSDLLIDENLLSPLAGEDDPFLLEGDVNEDCKPLILPDTKPKIQDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDNLTSLGAMNFAGRSVFSNGYSSPGVRPDVSSPPSSSSTATGPPPKLCLVCSDEA

SEQ ID NO: 71 - protein, 444 aa, Homo sapiens
MDSKESLTPPGRDEVPSSLLGRGRGSVMDLYKTLRGGATVKVSASSPSVAAASQADSKQQRILLDFSKGSASNAQQQQQQQQQQQQQQQQQPQPDLSKAISLSMGLYMGETETKVMGNDLGYPQQGQLGLSSGETDFRLLEESIANLNRSTSRPENPKSSTPAAGCATPTEKEFPQTHSDPSSEQQNRKSQPGTNGGSVKLYTTDQSTLDILQDLEFSAGSPGKETNESPWRSDLLIDENLLSPLAGEDDPFLLEGDVNEDCKPLILPDTKPKIQDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDNLTSLGAMNFAGRSVFSNGYSSPGMRPDVSSPPSSSSTATGPPPKLCLVCSDEA

(19) United States
(12) Patent Application Publication
(10) Pub. No.: US 2014/0213474 A1
(43) Pub. Date: Jul. 31, 2014

(54) GLUCOCORTICOID RECEPTOR ALLELES AND USES THEREOF

(71) Applicant: Shriners Hospitals for Children, Tampa, FL (US)

(72) Inventors: Kiho Cho, Davis, CA (US); David G. Greenhalgh, Davis, CA (US)

(73) Assignee: Shriners Hospitals for Children, Tampa, FL (US)

(21) Appl. No.: 14/204,787

(22) Filed: Mar. 11, 2014

Related U.S. Application Data
(63) Continuation of application No. 13/254,348, filed on Feb. 24, 2012, now abandoned.
(60) Provisional application No. 61/066,114, filed on Feb. 15, 2008.

(51) Int. Cl. C12Q 1/68; G01N 33/74

(52) U.S. Cl. CPC C12Q 1/6883; G01N 33/743
USPC 506/9; 435/6.11; 435/7.21; 514/169

(57) ABSTRACT

Provided here in are, inter alia, methods of determining whether a patient is resistant or sensitive to glucocorticoid therapy.

### RELATED APPLICATIONS

This application is a continuation of U.S. application Ser. No. 13/254,348, filed on Sep. 1, 2011, which is the National Stage of International Application No. PCT/US2009/034327, filed on Feb. 17, 2009, which claims priority under 35 U.S.C. §119(e) to U.S. Application No. 61/066,114, filed on Feb. 15, 2008, the entire contents of which are hereby incorporated by reference in their entireties.

### TECHNICAL FIELD

This invention relates, inter alia, to methods of identifying and categorizing patients according to their differing sensitivities to glucocorticoid therapy.

### BACKGROUND

Patients suffering from inflammatory disorders (e.g., asthma, COPD), burn wounds, and sepsis are routinely treated with glucocorticoids. However, a significant percentage of these patients do not respond, or are hypersensitive to such treatments. Currently, there is no good way to identify those patients who may be resistant or hypersensitive to glucocorticoids before the treatment is initiated.

The glucocorticoid receptor (GR) is a nuclear receptor involved in mediating anti-inflammatory responses. GRs include an N-terminal transactivation domain (t1), a central DNA-binding doman (DBD), a hinge region, and a C-terminal ligand-binding domain (LBD), which contains a second transactivation domain (t2). In the absence of hormone, GRs reside in the cytosol. The endogenous glucocortiod hormone cortisol diffuses through the cell membrane into the cytoplasm and binds to GRs. The resulting activated GRs homodimerize and translocate to the nucleus and bind to specific DNA sequences called the glucocorticoid response element (GRE) to activate gene transcription. Activated GRs can also bind to other transcription factors, such as NF-κB, and inhibit their ability to transactivate their target genes.

### SUMMARY

The invention is based, at least in part, on the discovery of a number of glucocorticoid receptor mutations in humans, and the use of these mutations to identify and categorize human subjects as to their sensitivity, e.g., resistance or hypersensitivity, to glucocorticoid therapy. In one aspect, the invention provides methods of determining whether a subject is glucocorticoid resistant or hypersensitive, the method including determining whether the subject has any one or more of the mutations of the GR and/or the GR gene as described herein.

The present disclosure provides methods of determining whether a subject is resistant to glucocorticoid treatment. The method can include obtaining a biological sample, e.g., a blood sample, from the subject, and analyzing the sample for the presence of a GR nucleic acid, e.g., comprising one or more mutations. The GR nucleic acid can encode a GR polypeptide with decreased transactivation potential as compared to a reference GR polypeptide comprising SEQ ID NO: 2. The presence of the GR nucleic acid indicates that the subject is resistant to glucocorticoid treatment.

For example, the presence of a GR nucleic acid that encodes a mutated GR polypeptide, e.g., a truncated GR polypeptide, a GR polypeptide that has a reduced ability to bind to a glucocorticoid, e.g., does not bind glucocorticoid, and/or a GR polypeptide with reduced transactivation potential, indicates that the subject is resistant to glucocorticoid treatment. In other cases, the presence of a GR nucleic acid including a G1379A mutation, e.g., encoding a GR polypeptide with a R460K amino acid substitution, as compared to SEQ ID NO: 1 indicates that the subject is resistant to glucocorticoid treatment. In some instances, the presence of a GR nucleic acid including a T2246C mutation, e.g., encoding a GR polypeptide with a F749G amino acid substitution, as compared to SEQ ID NO: 1, indicates that the subject is resistant to glucocorticoid treatment. The presence of a GR nucleic acid with 17 CAG repeats in exon 2, e.g., encoding a GR polypeptide comprises 17 glutamine repeats in the transactivation domain of the GR polypeptide, can also indicate that the subject is resistant to glucocorticoid treatment. In another example, the GR nucleic acid can encode a GR polypeptide with one or more mutations in the DNA binding domain such that GR polypeptide has decreased transactivation potential, e.g., decreased responsiveness to glucocorticoid.

The method can further include administering an agent or a treatment other than glucocorticoid, or increased dosages of glucocorticoid, to the subject if the subject is identified to have a GR nucleic acid with one or more mutations and encoding a GR polypeptide with decreased transactivation potential.

Included herein are methods of determining whether a subject is hypersensitive to glucocorticoid treatment. The method can include obtaining a biological sample from the subject and analyzing the sample for the presence of a GR nucleic acid including one or more mutations and encoding a GR polypeptide with increased transactivation potential as compared to a reference GR polypeptide comprising SEQ ID NO: 2. The presence of the GR nucleic acid indicates that the subject is hypersensitive to glucocorticoid treatment. In some instances, the GR nucleic acid comprises a A2297G mutation. In other instances, the GR nucleic acid encodes a polypeptide having a N766S amino acid substitution.

The method can further include modifying glucocorticoid treatment of the subject, e.g., administering a low dosage of glucocorticoid to the patient, or administering an agent or a treatment other than glucocorticoid to the subject, if the subject is identified to have a GR nucleic acid with one or more mutations and encoding a GR polypeptide with increased transactivation potential.

In some instances, detecting a GR nucleic acid with one or more mutations described herein can include isolating genomic DNA from a sample, performing polymerase chain reaction (PCR) on the genomic DNA using a primer selected from the group consisting of SEQ ID NOs: 3-61, and sequencing the PCR product. In other instances, detecting can include detecting a GR polypeptide using an antibody that binds specifically to the mutant GR polypeptide. In still other instances, detecting can include detecting a GR nucleic acid and a GR polypeptide. Other art-known can be used in the methods described herein to determine whether a subject has a GR nucleic acid, e.g., with one or more of the mutations described herein.

In some cases, whether a patient has a mutated GR can be determined by detecting a mutation in a nucleic acid sequence, e.g., the GR gene or a portion thereof, that encodes the receptor, detecting a mutation in a GR polypeptide, or both.

Also provided herein are nucleic acid molecules, e.g., isolated and/or purified nucleic acid molecules having one or more of the mutations described herein. These nucleic acid molecules can encode, e.g., a mutated glucocorticoid receptor (GR) polypeptide having an altered transactivation potential. For example, the isolated and purified nucleic acid molecule can encode a truncated GR polypeptide. The truncated GR polypeptide can, e.g., lack a ligand binding domain. Alternatively, the mutated GR polypeptide can have an altered ligand binding domain, e.g., one that exhibits a reduced affinity for glucocorticoid. In other instances, the isolated and purified nucleic acid molecule can encode a GR polypeptide with one or more of the mutations described herein, e.g., a F749G amino acid substitution, a N766S amino acid substitution, a R460K amino acid substitution, and a 17-glutamine repeat.

Isolated nucleic acid molecules encoding fusion proteins are also provided. For example, the nucleic acid can include a first nucleic acid sequence that encodes a fragment of a GR polypeptide and a second nucleic acid sequence linked to the first that encodes a non-GR polypeptide. In some instances, the isolated nucleic acid molecules comprise a first sequence that encodes a GR polypeptide that lacks a ligand binding domain or a portion thereof. In others, the isolated nucleic acid molecules can comprise a first sequence that encodes a GR polypeptide that has decreased transactivation potential, e.g., reduced affinity for glucocorticoid, as compared to a reference GR polypeptide. In other instances, the isolated nucleic acid molecules can comprise a first sequence that encodes a GR polypeptide that has increased transactivation potential as compared to a reference GR polypeptide. The second nucleic acid sequence can encode a non-GR polypeptide, e.g., a hexa-histidine tag, FLAG tag, a hemagglutinin tag, an immunoglobulin constant (Fc) region or a detectable marker (e.g., β-galactosidase, invertase, green fluorescent protein, luciferase, chloramphenicol acetyltransferase, beta-glucuronidase, exo-glucanase, and/or glucoamylase).

An isolated vector comprising a nucleic acid sequence described herein is also provided. For example, the nucleic acid sequence can encode a mutated GR polypeptide.

Also provided herein are recombinant polypeptides encoded by the nucleic acid sequences described herein. In some cases, the recombinant polypeptides include a fragment of GR polypeptide and a non-GR polypeptide.

Also provided herein is an anti-GR antibody that specifically binds a GR polypeptide described herein. In certain cases, the anti-GR antibody binds to a mutated GR polypeptide, e.g., a GR polypeptide lacking a ligand binding domain or a portion thereof.

Also provided herein are a first primer and a second primer, wherein the first primer is selected from SEQ ID NOs: 3-61, and the second primer is selected from SEQ ID NOs: 3-61 and wherein the first and second primers are not the same primer.

The instant disclosure also provides kits, e.g., for determining whether a subject is resistant or hypersensitive to glucocorticoid treatment. The kits can include means for detecting whether the subject has a GR nucleic acid comprising one or more mutations, and instructional material for using the kits.

In one embodiment, the kit includes one or more pairs of primers for amplifying a specific region, e.g., exon 2 and exon 9α, of the GR gene. Each pair of the primers can include two different primers flanking a region of interest, e.g., a forward primer and a reverse primer listed in Table 1. Each pair of primers can be used to PCR amplify a region of the GR gene from a subject, and the PCR products can be sequenced to determine whether the subject has a GR nucleic acid with one or more mutations described herein, e.g., a A2297G mutation.

In another embodiment, the kit includes one or more antibodies that bind specifically to a GR polypeptide, e.g., one with a N766S amino acid substitution, encoded by a GR nucleic acid comprising one or more mutations. The antibodies can be used to detect the presence of the GR polypeptide by, e.g., Western blotting.

In other embodiments, the kit includes one or more microarrays with oligonucleotides that hybridize to specific GR nucleic acid sequences, e.g., GR nucleic acid sequences containing one or more mutations described herein.

Instruction material in the kit can include instructions for treating subjects determined to have a GR nucleic acid having one or more mutations, e.g., subjects identified to be resistant or hypersensitive to glucocorticoid treatment. For example, the instruction material can include instructions for treating subjects who are determined to be glucocorticoid resistant with non-glucocorticoid anti-inflammatory agents, e.g., an agent described herein. In another example, the instruction material can include instructions for modifying a glucocorticoid treatment for those subjects who are determined to be glucocorticoid hypersensitive.

The kits can further include reagents, e.g., buffers, enzymes and plasmids, and other material for using the kits.

The invention also provides kits that include a device, e.g., wherein a GR nucleic acid sequence with one or more mutations is detected by oligonucleotides or antibodies, and instruction material for using the kits.

Also provided herein are methods of identifying a candidate compound for treating inflammation in a patient with a glucocorticoid receptor (GR) allele. For example, the method can include contacting in vitro a GR polypeptide encoded by the GR allele and a test compound; and determining whether the test compound modulates an activity of the GR polypeptide; wherein a test compound that modulates the activity of the GR polypeptide is a candidate compound for treating inflammation in the patient. The methods can involve determining whether a test compound modulates a GR polypeptide's activity, e.g., to bind to a glucocorticoid, to homoderimerize, to translocate into the nucleus, to bind to a nucleic acid (e.g., to a GRE), to bind to other transcription factors, or to induce or transactivate transcription of a gene. In some cases, a test compound that can modulate the transactivation potential of a GR polypeptide encoded by a GR allele is a candidate compound for treating inflammation in a patient having the GR allele. The methods can be carried out in cells, e.g., human embryonic kidney cells, or cell-free systems. For example, the methods can be carried out in cells that express a mutated GR polypeptide, wherein test compounds are screened for their ability to bind to and/or activate the GR polypeptide.

The invention also provides methods of identifying a candidate compound, the method comprising: providing a cell expressing a GR polypeptide that is not responsive to a glucocorticoid; administering a test compound to the cell; and detecting an anti-inflammatory response in the cell, e.g., expression of an anti-inflammatory gene, wherein a test compound that induces an anti-inflammatory response in the cell is a candidate compound. A GR polypeptide that is not responsive to a glucocorticoid can be, for example, a truncated GR polypeptide (e.g., one lacking, in whole or in part, a ligand binding domain).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

FIG. 1 is a nucleic acid sequence of a human glucocorticoid receptor nucleic acid (SEQ ID NO:1).

FIG. 2 is an amino acid sequence of a human glucocorticoid receptor polypeptide (SEQ ID NO:2).

### DETAILED DESCRIPTION

The invention is based, at least in part, on the discovery of certain GR mutations in humans and mice. Patients having GR alleles described herein, e.g., ones that encode GR polypeptides lacking the ligand binding domain (LBD), are expected to cause a patient to be resistant, e.g., non-responsive, or partially resistant, to glucocorticoid treatment. Accordingly, provided herein are, inter alia, GR nucleic acids and polypeptides that represent specific GR mutations (in the GR gene and/or polypeptide), methods of identifying and categorizing glucocorticoid resistant or hypersensitive patients, methods of identifying novel compounds using recombinant GR proteins described herein, and kits for identifying and categorizing glucocorticoid resistant or hypersensitive patients.

### I. Nucleic Acids, Proteins, Vectors, and Host Cells

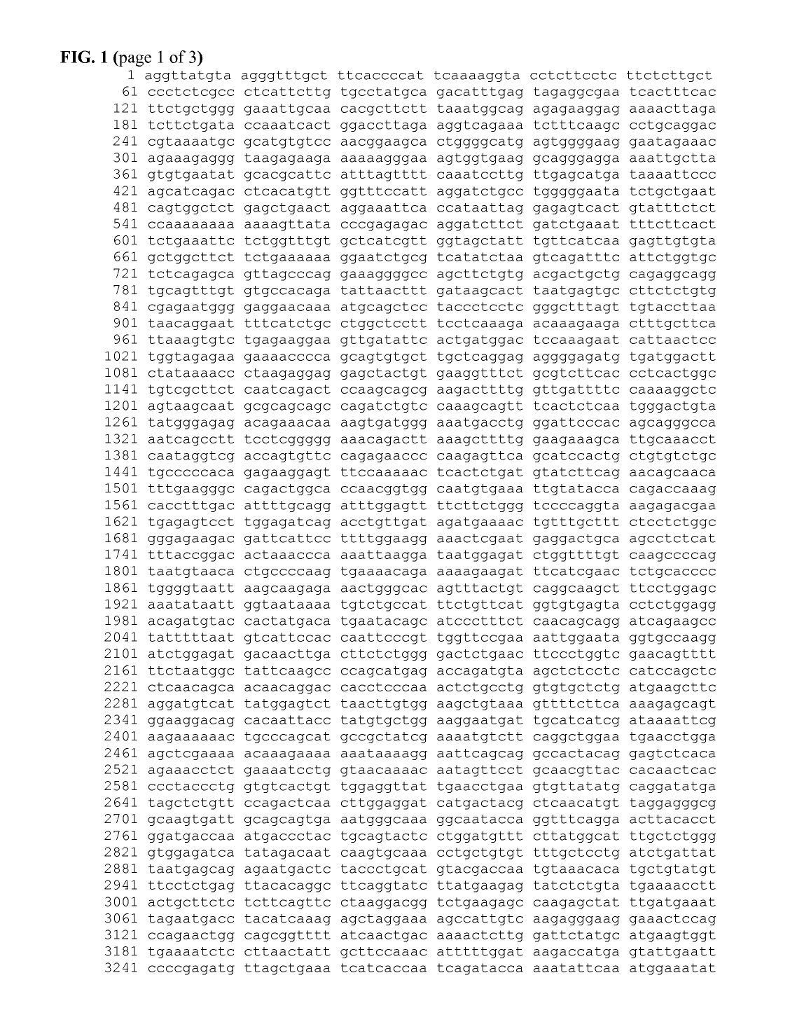

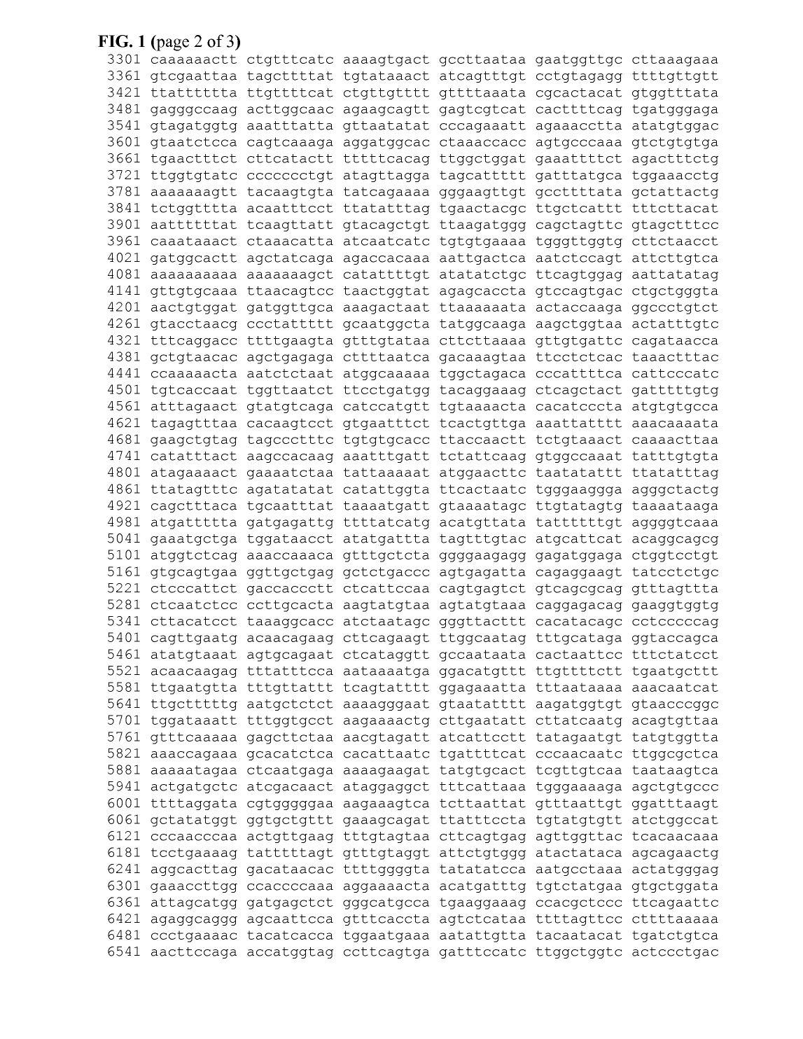

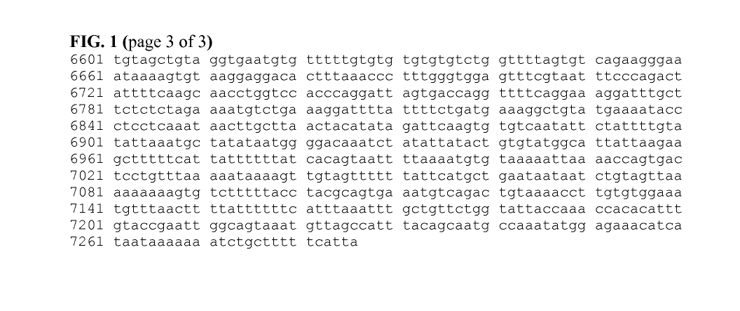

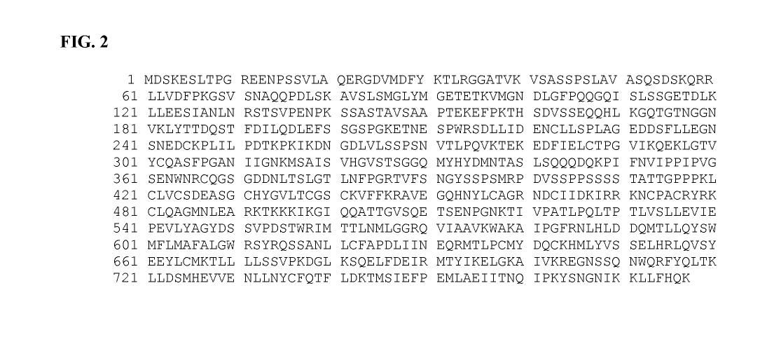

In one aspect, the invention includes certain isolated and/or recombinant GR nucleic acids. Full-length GR nucleic acids include human GR nucleic acid sequence, such as SEQ ID NO: 1 (GenBank Accession No. NM_{—}001018077; shown in FIG. 1; FIG. 2 is the amino acid sequence of a human glucocorticoid receptor polypeptide). SEQ ID NO: 1 is also referred to herein as the reference human GR nucleic acid sequence.

A recombinant GR nucleic acid can include a fragment of a GR nucleic acid, e.g., a fragment of SEQ ID NO: 1. A fragment of a GR nucleic acid encodes at least one useful fragment of a GR polypeptide (e.g., a human or rodent polypeptide), e.g., a fragment containing an N-terminal domain, a DNA binding domain, and/or a ligand binding domain, or other useful fragment. For example, a useful fragment of a GR nucleic acid may encode a fragment of a GR polypeptide capable of binding a compound, e.g., e.g., an intact DNA binding domain, e.g., a fragment corresponding to amino acids from about 1 to about 490 of SEQ ID NO: 2 (the amino acid sequence of a polypeptide encoded by SEQ ID NO: 1). As another example, a useful fragment of a GR nucleic acid may encode a fragment of a GR polypeptide lacking at least part of a ligand binding domain, e.g., a fragment corresponding to amino acids from about 1 to about 550 of SEQ ID NO:2. Other useful GR nucleic acid fragments are those containing one or more mutations, e.g., the mutations described herein, and encode GR polypeptides with one or more mutations that, e.g., exhibit deceased or enhanced transactivation potential.

The GR nucleic acids described herein include both RNA and DNA, including genomic DNA and synthetic (e.g., chemically synthesized) DNA. Nucleic acids can be double-stranded or single-stranded. Where single-stranded, the nucleic acid can be a sense strand or an antisense strand. Nucleic acids can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

The term “isolated nucleic acid” means a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated GR nucleic acid includes some or all of the 5′ non-coding (e.g., promoter) sequences that are immediately contiguous to the GR nucleic acid coding sequence. The term includes, for example, recombinant DNA that is incorporated into a vector, into an autonomously replicating plasmid or virus, or into the genomic DNA of a prokaryote or eukaryote, or which exists as a separate molecule (e.g., a genomic DNA fragment produced by PCR or restriction endonuclease treatment) independent of other sequences. It also includes a recombinant DNA that is part of a hybrid gene encoding an additional polypeptide sequence.

The term “purified” refers to a GR nucleic acid (or GR polypeptide) that is substantially free of cellular or viral material with which it is naturally associated, or culture medium (when produced by recombinant DNA techniques), or chemical precursors or other chemicals (when chemically synthesized). Moreover, an isolated nucleic acid fragment is a nucleic acid fragment that is not naturally occurring as a fragment and would not be found in the natural state.

In some instances, the invention includes nucleic acid sequences that are substantially identical to a GR nucleic acid. A nucleic acid sequence that is “substantially identical” to a GR nucleic acid is at least 75% identical (e.g., at least about 80%, 85%, 90%, or 95% identical) to the GR nucleic acid sequences represented by SEQ ID NO: 1. For purposes of comparison of nucleic acids, the length of the reference nucleic acid sequence will typically be at least 50 nucleotides, but can be longer, e.g., at least 60 nucleotides, or more nucleotides.

To determine the percent identity of two amino acid or nucleic acid sequences, the sequences are aligned for optimal comparison purposes (i.e., gaps can be introduced as required in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of overlapping positions×100). The two sequences can be of the same length.

The percent identity or homology between two sequences is determined using the mathematical algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990); J. Mol. Biol. 215:403-410. BLAST nucleotide searches are performed with the NBLAST program, score=100, wordlength=12 to obtain nucleotide sequences homologous to GR nucleic acid molecules of the invention. BLAST protein searches are performed with the XBLAST program, score=50, wordlength=3 to obtain amino acid sequences homologous to GR protein molecules of the invention. To obtain gapped alignments for comparison purposes, Gapped BLAST is utilized as described in Altschul et al., (1997) Nucleic Acids Res. 25:3389-3402. When utilizing BLAST and Gapped BLAST programs, the default parameters of the respective programs (e.g., XBLAST and NBLAST) are used. See online at ncbi.nlm.nih.gov.

The invention also includes variants, homologs, and/or fragments of a reference GR nucleic acid, e.g., variants, homologs, and/or fragments of the GR nucleic acid sequence represented by SEQ ID NO:1. The terms “variant” or “homolog” in relation to GR nucleic acids include any substitution, variation, modification, replacement, deletion, or addition of one (or more) nucleotides from or to the sequence of a reference GR nucleic acid. The resultant nucleotide sequence may encode a GR polypeptide that is generally at least as biologically active as the referenced GR polypeptide (e.g., as represented by SEQ ID NO: 2). In particular, the term “homolog” covers homology with respect to structure and/or function provided that the resultant nucleotide sequence codes for or is capable of coding for a GR polypeptide being at least as biologically active as GR encoded by a sequence shown herein as SEQ ID NO:2. With respect to sequence homology, there is at least 75% (e.g., 85%, 90%, 95%, 98%, or 100%) homology to the sequence shown as SEQ ID NO: 1.

Also included within the scope of the present invention are certain alleles of the GR gene. As used herein, an “allele” or “allelic sequence” is an alternative form of GR. Alleles result from a mutation, i.e., a change in the nucleotide sequence, and generally produce altered mRNAs or polypeptides whose structure or function may or may not be altered. Any given gene can have none, one, or more than one allelic form. Common mutational changes that give rise to alleles are generally ascribed to deletions, additions, or substitutions of amino acids. Each of these types of changes can occur alone, or in combination with the others, one or more times in a given sequence. A GR allele or a GR allelic sequence contains one or more mutational changes as compared to SEQ ID NO: 1, or the reference human GR nucleic acid sequence. For example, a GR allele can have a mutation, e.g., one or more of the mutations listed in Tables 2, 3A, 3B, 4 and 5 below, resulting in a premature stop codon such that the allele encodes a truncated GR polypeptide, e.g., a fragment of a GR polypeptide lacking at least part of a ligand binding domain. A GR allele can also encode a full-length GR polypeptide containing one or more amino acid substitutions, e.g., one or more of the amino acid substitutions listed in Tables 2, 3A, 3B, 4 and 5, as compared to SEQ ID NO: 2, the reference human GR amino acid sequence.

The invention also includes nucleic acids that hybridize, e.g., under stringent hybridization conditions (as defined herein) to all or a portion of the nucleotide sequences represented by SEQ ID NO: 1. The hybridizing portion of the hybridizing nucleic acids is typically at least 15 (e.g., 20, 30, or 50) nucleotides in length. The hybridizing portion of the hybridizing nucleic acid is at least about 75%, e.g., at least about 80%, 95%, 98% or 100%, identical to the sequence of a portion or all of a nucleic acid encoding an GR polypeptide, or to its complement. Hybridizing nucleic acids of the type described herein can be used as a cloning probe, a primer (e.g., a PCR primer), or a diagnostic probe.

High stringency conditions are hybridizing at 68° C. in 5×SSC/5×Denhardt's solution/1.0% SDS, or in 0.5 M NaHPO₄ (pH 7.2)/1 mM EDTA/7% SDS, or in 50% formamide/0.25 M NaHPO₄ (pH 7.2)/0.25 M NaC1/1 mM EDTA/7% SDS; and washing in 0.2×SSC/0.1% SDS at room temperature or at 42° C., or in 0.1×SSC/0.1% SDS at 68° C., or in 40 mM NaHPO₄ (pH 7.2)/1 mM EDTA/5% SDS at 50° C., or in 40 mM NaHPO₄ (pH 7.2) 1 mM EDTA/1% SDS at 50° C. Stringent conditions include washing in 3×SSC at 42° C. The parameters of salt concentration and temperature can be varied to achieve the optimal level of identity between the probe and the target nucleic acid. Additional guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The invention also provides primers that hybridize to portions of a GR nucleic acid, e.g., portions of SEQ ID NO: 1 or a mouse GR gene. These primers can be used, e.g., to amplify a specific region in the GR gene that may contain one or more mutations, e.g., those mutations described herein. The sequences of these primers are shown below in Table 1:

TABLE 1

| | | |
|---|---|---|
| SEQ ID NO: 3 (2-1A) | Exon 2 forward primer | ccagcagtgtgcttgctca |
| SEQ ID NO: 4 (2-1B) | Exon 2 forward primer | cagactccaagcagcgaaga |
| SEQ ID NO: 5 (2-2A) | Exon 2 reverse primer | ccagaggtactcacaccatgaac |
| SEQ ID NO: 6 (2-2B) | Exon 2 reverse primer | ccagggaagttcagagtcc |
| SEQ ID NO: 7 (2-2C) | Exon 2 reverse primer | gccaccgttggtgccagtctg |
| SEQ ID NO: 8 (2-2D) | Exon 2 reverse primer | gtcaaaggtgctttggtctgtgg |
| SEQ ID NO: 9 (3-1A) | Exon 3 forward primer | ccagcatgagaccagatgta |
| SEQ ID NO: 10 (3-2A) | Exon 3 reverse primer | aagcttcatcagagcacacc |
| SEQ ID NO: 11 (3-2B) | Exon 3 reverse primer | cttccactgctcttttgaagaa |
| SEQ ID NO: 12 (4-1A) | Exon 4 forward primer | gacagcacaattacctatgtgctg |
| SEQ ID NO: 13 (4-1B) | Exon 4 forward primer | cagcacaattacctatgtgctgga |
| SEQ ID NO: 14 (4-2A) | Exon 4 reverse primer | cttccaggttcattccagcctgaa |
| SEQ ID NO: 15 (4-2B) | Exon 4 reverse primer | ccaggttcattccagcctgaagac |
| SEQ ID NO: 16 (5-1A) | Exon 5 forward primer | ggaattcagcaggccactacagg |
| SEQ ID NO: 17 (5-1B) | Exon 5 forward primer | caggccactacaggagtctc |
| SEQ ID NO: 18 (5-2A) | Exon 5 reverse primer | ctggtattgcctttgcccatttc |
| SEQ ID NO: 19 (6-1A) | Exon 6 forward primer | gtttcaggaacttacacctggatg |
| SEQ ID NO: 20 (6-2A) | Exon 6 reverse primer | cacagcaggtttgcac |
| SEQ ID NO: 21 (6-2B) | Exon 6 reverse primer | tcattaataatcagatcaggagc |
| SEQ ID NO: 22 (7-1A) | Exon 7 forward primer | gcagagaatgactctaccctgca |
| SEQ ID NO: 23 (7-1B) | Exon 7 forward primer | cctgcatgtacgaccaatg |
| SEQ ID NO: 24 (7-2A) | Exon 7 reverse primer | gagagaagcagtaagg |
| SEQ ID NO: 25 (7-2B) | Exon 7 reverse primer | cctgaagagagaagcagtaa |
| SEQ ID NO: 26 (8-1A) | Exon 8 forward primer | tcctaaggacggtctgaagagcc |
| SEQ ID NO: 27 (8-2A) | Exon 8 reverse primer | aaccgctgccagttctggctgga |
| SEQ ID NO: 28 (8-2B) | Exon 8 reverse primer | ttcatgcatagaatccaagag |
| SEQ ID NO: 29 (9a-1A) | Exon 9α forward primer | tacgcactacatgtgg |
| SEQ ID NO: 30 (9a-1B) | Exon 9α forward primer | tggcaacagaagcagttgag |
| SEQ ID NO: 31 (9a-1C) | Exon 9α forward primer | cagctgtttaagatgggcagc |
| SEQ ID NO: 32 (9a-1D) | Exon 9α forward primer | ccagataaccagctgtaacacagc |
| SEQ ID NO: 33 (9a-1E) | Exon 9α forward primer | cacattcccatctgtcacca |
| SEQ ID NO: 34 (9a-1F) | Exon 9α forward primer | ccactgaccaatttggaagcc |
| SEQ ID NO: 35 (9a-2A) | Exon 9α reverse primer | tgggtcagagcctcagcaa |
| SEQ ID NO: 36 (9a-2B) | Exon 9α reverse primer | ggagggctgtatgtgaaag |
| SEQ ID NO: 37 (9a-2C) | Exon 9α reverse primer | ccacatgtagtgcgta |
| SEQ ID NO: 38 (9b-1A) | Exon 9β forward primer | catcccaacaatcttggc |
| SEQ ID NO: 39 (9b-1B) | Exon 9β forward primer | gctcatcgacaactataggagg |
| SEQ ID NO: 40 (9b-1D) | Exon 9β forward primer | gtgcagaatctcataggttgcc |
| SEQ ID NO: 41 (9b-2A) | Exon 9β reverse primer | aaacaggagtcactgg |
| SEQ ID NO: 42 (9b-2B) | Exon 9β reverse primer | ctgccaattcggtaca |
| SEQ ID NO: 43 (9b-2C) | Exon 9β reverse primer | cctcctatagttgtcgatgagc |
| SEQ ID NO: 44 (1A) | Exon 2 forward primer | atattcactgatggactcca |
| SEQ ID NO: 45 (1B) | Exon 2 forward primer | tcactgatggactccaaag |
| SEQ ID NO: 46 (1C) | Exon 2 forward primer | ttcactgatggactccaaagaatcattaac |
| SEQ ID NO: 47 (1D) | Exon 2 forward primer | tgatattcactgatggactccaaagaatca |
| SEQ ID NO: 48 (1E) | Exon 2 forward primer | gagcggctcctctgccag |
| SEQ ID NO: 49 (1F) | Exon 2 forward primer | gctcctctgccagagttg |
| SEQ ID NO: 50 (1G) | Exon 2 forward primer | gaactgcggacggtgg |
| SEQ ID NO: 51 (1H) | Exon 2 forward primer | tgcggcgggaactgcg |
| SEQ ID NO: 52 (a-2A) | Exon 9α reverse primer | ttaaggcagtcacttttgatgaaac |
| SEQ ID NO: 53 (a-2B) | Exon 9α reverse primer | ccattcttattaaggcagtca |
| SEQ ID NO: 54 (a-2C) | Exon 9α reverse primer | ttattaaggcagtcacttttgatgaaacag |
| SEQ ID NO: 55 (a-2D) | Exon 9α reverse primer | aggcaaccattcttattaaggcagtcactt |
| SEQ ID NO: 56 (a-2E) | Exon 9α reverse primer | cctctacaggacaaactga |
| SEQ ID NO: 57 (a-2F) | Exon 9α reverse primer | caacaaaacctctaca |
| SEQ ID NO: 58 (GR1B) | mGR Exon 1 forward primer | ccaagcagcagaggattctcc |
| SEQ ID NO: 59 (mGR1-1F) | mGR Exon 1 forward primer | cagcagcaccgcagccagattta |
| SEQ ID NO: 60 (hGR2-2D) | Exon 2 reverse primer | gtcaaaggtgctttggtctgtgg |
| SEQ ID NO: 61 (hGR2-2E) | Exon 2 reverse primer | ccaaggactctcattcgtctc |

Skilled practitioners will appreciate that certain modifications can be made to the above-referenced primers without substantially changing the hybridizing/priming activity of the primers. Such modified primers are within the present invention.

Also included in the invention are genetic constructs (e.g., vectors and plasmids) that include a GR nucleic acid described herein, operably linked to a transcription and/or translation sequence to enable expression, e.g., expression vectors. A selected nucleic acid, e.g., a DNA molecule encoding a GR polypeptide, is “operably linked” to another nucleic acid molecule, e.g., a promoter, when it is positioned either adjacent to the other molecule or in the same or other location such that the other molecule can direct transcription and/or translation of the selected nucleic acid. These genetic constructs are useful for, e.g., the screening methods described herein or testing the transactivation potential of a GR polypeptide.

Also included in the invention are various engineered cells, e.g., transformed host cells, which contain a GR nucleic acid described herein. A transformed cell is a cell into which (or into an ancestor of which) has been introduced, by means of standard techniques, a nucleic acid encoding a GR polypeptide. Both prokaryotic and eukaryotic cells are included, e.g., mammalian cells (e.g., osteoblasts) fungi (such as yeast), and bacteria (such as Escherichia coli), and the like.

Certain recombinant GR polypeptides and isolated fragments of GR polypeptides are also included within the present invention. An exemplary full-length GR polypeptide is the human

GR polypeptide shown in SEQ ID NO: 2, or a human GR polypeptide containing one or more of the amino acid substitutions described herein.

Included within the present invention are GR polypeptides encoded by the GR nucleic acids described herein. Also included within the present invention are certain fragments of GR polypeptides, e.g., fragments of SEQ ID NO: 2. Fragments of GR polypeptides may include a N-terminal transactivation domain, a DNA binding domain, and/or other useful portion of a full-length GR polypeptide. For example, useful fragments of GR polypeptides include, but are not limited to, fragments lacking a ligand binding domain (e.g., a fragment including amino acids about 1 to about 490 of SEQ ID NO: 2) and portions of such fragments.

The terms “protein” and “polypeptide” both refer to any chain of amino acids, regardless of length or post-translational modification (e.g., glycosylation or phosphorylation). Thus, the terms “GR protein” and “GR polypeptide” include full-length naturally occurring isolated proteins, as well as recombinantly or synthetically produced polypeptides that correspond to the full-length naturally occurring proteins, or to a fragment of the full-length naturally occurring or synthetic polypeptide.

As discussed above, the term “GR polypeptide” includes both biologically active and non-biologically active fragments of naturally occurring or synthetic GR polypeptides. Fragments of a protein can be produced by any of a variety of methods known to those skilled in the art, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid that encodes the polypeptide. Expression of such mutagenized DNA can produce polypeptide fragments. Digestion with “end-nibbling” endonucleases can thus generate DNAs that encode an array of fragments. DNAs that encode fragments of a protein can also be generated, e.g., by random shearing, restriction digestion, chemical synthesis of oligonucleotides, amplification of DNA using the polymerase chain reaction, or a combination of the above-discussed methods. Fragments can also be chemically synthesized using techniques known in the art, e.g., conventional Merrifield solid phase FMOC or t-Boc chemistry. For example, peptides of the present invention can be arbitrarily divided into fragments of desired length with no overlap of the fragments, or divided into overlapping fragments of a desired length.

A purified or isolated compound is a composition that is at least 60% by weight the compound of interest, e.g., a GR polypeptide or antibody. Typically, the preparation is at least 75% (e.g., at least 90%, 95%, or 99%) by weight the compound of interest. Purity can be measured by any appropriate standard method, e.g., column chromatography, polyacrylamide gel electrophoresis, or HPLC analysis.

In certain embodiments, GR polypeptides include sequences substantially identical to all or portions of a naturally occurring GR polypeptides. Polypeptides “substantially identical” to the GR polypeptide sequences described herein have an amino acid sequence that is at least 65% (e.g., at least 75%, 80%, 85%, 90%, 95%, 99% or 99.9%, e.g., 100%), identical to the amino acid sequences of the GR polypeptides represented by SEQ ID NO:2 (measured as described herein). For purposes of comparison, the length of the reference GR polypeptide sequence is typically at least 16 amino acids, e.g., at least 20 or 25 amino acids.

In the case of polypeptide sequences that are less than 100% identical to a reference sequence, the non-identical positions are preferably, but not necessarily, conservative substitutions for the reference sequence. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Where a particular polypeptide is said to have a specific percent identity to a reference polypeptide of a defined length, the percent identity is relative to the reference polypeptide. Thus, a polypeptide that is 50% identical to a reference polypeptide that is 100 amino acids long can be a 50 amino acid polypeptide that is completely identical to a 50 amino acid long portion of the reference polypeptide. It also can be, e.g., a 100 amino acid long polypeptide that is 50% identical to the reference polypeptide over its entire length.

GR polypeptides of the invention include, but are not limited to, recombinant polypeptides and natural polypeptides. Also included are nucleic acid sequences that encode forms of GR polypeptides in which naturally occurring amino acid sequences are altered or deleted. Certain nucleic acids of the present invention may encode polypeptides that are soluble under normal physiological conditions.

Also within the invention are nucleic acids encoding fusion proteins, and the fusion proteins themselves, in which a GR polypeptide is fused to an unrelated polypeptide, also referred to herein as a “heterologous polypeptide” or a “non-GR polypeptide” (e.g., a marker polypeptide or a fusion partner) to create a fusion protein. For example, the polypeptide can be fused to a hexa-histidine tag or a FLAG tag to facilitate purification of bacterially expressed polypeptides or to a hemagglutinin tag or a FLAG tag to facilitate purification of polypeptides expressed in eukaryotic cells. The invention also includes, for example, isolated polypeptides (and the nucleic acids that encode these polypeptides) that include a first portion and a second portion, where the first portion includes, e.g., a GR polypeptide, and the second portion includes an immunoglobulin constant (Fc) region or a detectable marker (e.g., β-galactosidase, invertase, green fluorescent protein, luciferase, chloramphenicol acetyltransferase, beta-glucuronidase, exo-glucanase, and/or glucoamylase).

The fusion partner can be, for example, a polypeptide that facilitates secretion, e.g., a secretory sequence. Such a fused polypeptide is typically referred to as a preprotein. The secretory sequence can be cleaved by the host cell to form the mature protein. Also within the invention are nucleic acids that encode a GR polypeptide fused to a polypeptide sequence to produce an inactive preprotein. Preproteins can be converted into the active form of the protein by removal of the inactivating sequence.

In some instances, it may be useful to determine whether a GR polypeptide is functional, e.g., whether the GR polypeptide has an activity. GR activities can include, but are not limited to, its ability to bind to a glucocorticoid, to homoderimerize, to translocate into the nucleus, to bind to a nucleic acid (e.g., to a GRE), to bind to other transcription factors, or to induce or transactivate transcription of a gene. As used herein, the term “transactivation potential” refers generally to a GR's ability to modulate, reduce, inhibit, induce, stimulate or transactivate the transcription of a gene, e.g., a gene under the control of a GRE or other genes modulated by GR. A GR polypeptide's transactivation potential can be altered or affected, for example, by its ability to bind to a glucocorticoid, to bind to a nucleic acid, or to transactivate a gene, e.g., its ability to interact with other components involved in gene transcription. Skilled practitioners will appreciate that conventional assays can be used to determined whether a GR polypeptide exhibits any of these activities. For example, in vitro or in vivo assays known in the art can be used to determine whether a GR polypeptide binds to a glucocorticoid. Conventional assays can also be used to determine whether a GR polypeptide binds to a nucleic acid molecule or a sequence.

An exemplary assay for determining the transactivation potential of a GR polypeptide is described below. The GR nucleic acid sequence encoding the GR polypeptide of interest can be cloned into a first vector (e.g., pcDNA4, Invitrogen, Carlsbad, Calif.). A second vector can be constructed to contain a reporter gene, e.g., the luciferase gene, under the control of a GRE. These two vectors can be co-transfected into a cell, e.g., a human embryonic kidney cell (HEK 293). Skilled practitioners will recognize that a number of cell types can be used to test whether a GR polypeptide is functional. The cell will then express the GR polypeptide encoded by the GR nucleic acid sequence on the first vector. The cell can be grown in media containing a basal level of glucocorticoid, e.g., media with fetal bovine serum. If the GR polypeptide is functional, it will bind to the GRE and transactivate the transcription of the reporter gene. The product of the reporter gene, e.g., luciferase, can then be detected and quantified using conventional methods. For example, when a luciferin substrate is added, luciferase will produce luminescence that can be detected and quantified. The amount of bioluminescence produced is therefore proportional to the amount of luciferase produced and, consequently, to the activation potential of the GR protein expressed in the cell. Other methods known in the art can be used to assay GR transactivation potential.

Certain GR alleles described herein encode GR polypeptides having decreased transactivativation potential as compared to the reference GR, e.g., GRs that lack a portion of the ligand binding domain, that include one or more mutations in the transactivation domain, DNA binding or ligand binding domain, and that include a transactivation domain that shares homology with a mouse GR transactivation domain. Other GR alleles described herein contain mutations, e.g., A2297G, that result in GR polypepitdes with increased or enhanced transactivation potential as compared to the reference GR.

### II. Antibodies

The invention features purified or isolated antibodies, i.e., anti-GR antibodies, that bind, e.g., specifically bind, to a GR polypeptide. An antibody “specifically binds” to a particular antigen, e.g., a GR polypeptide, when it binds to that antigen, and binds to a lesser extent (e.g., with lower affinity or not at all) to other molecules in a sample, e.g., a biological sample that includes a GR polypeptide. The antibodies described herein include monoclonal antibodies, polyclonal antibodies, humanized or chimeric antibodies, single chain antibodies, Fab fragments, F(ab′)₂ fragments, and molecules produced using a Fab expression library.

An example of a type of antibody included in the present invention is the polyclonal anti-GR antibody described herein. Such an antibody can be produced as follows: a peptide corresponding to GR amino acid residues about 1 to about 425 (e.g., of SEQ ID NO:2), inclusive, is synthesized, coupled to ovalbumin, and injected into rabbits to raise rabbit polyclonal antibodies.

As used herein, the term “antibody” refers to a protein comprising at least one, e.g., two, heavy (H) chain variable regions (abbreviated herein as VH), and at least one, e.g., two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Each VH and VL is composed of three CDR's and four FRs, arranged from amino-terminus to carboxy-terminus in the following order: FR1, CDR1, FR2, CDR2, FR3, CDR3, FR4.

An anti-GR antibody can further include a heavy and light chain constant region, to thereby form a heavy and light immunoglobulin chain, respectively. The antibody can be a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2, and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

A “GR binding fragment” of an antibody refers to one or more fragments of a full-length antibody that retain the ability to specifically bind to a GR polypeptide or a portion thereof. Examples of GR polypeptide binding fragments of an anti-GR antibody include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “GR binding fragment” of an antibody. These antibody fragments can be obtained using conventional techniques known to those with skill in the art.

To produce antibodies, GR polypeptides (or antigenic fragments (e.g., fragments of GR that appear likely to be antigenic by criteria such as high frequency of charged residues) or analogs of such polypeptides), e.g., those produced by standard recombinant or peptide synthetic techniques (see, e.g., Ausubel et al., supra), can be used. In general, the polypeptides can be coupled to a carrier protein, such as KLH, as described in Ausubel et al., supra, mixed with an adjuvant, and injected into a host mammal. A “carrier” is a substance that confers stability on, and/or aids or enhances the transport or immunogenicity of, an associated molecule. For example, nucleic acids encoding GR or fragments thereof can be generated using standard techniques of PCR, and can be cloned into a pGEX expression vector (Ausubel et al., supra). Fusion proteins can be expressed in E. coli and purified using a glutathione agarose affinity matrix as described in Ausubel, et al., supra.

Typically, to produce antibodies, various host animals are injected with GR polypeptides. Examples of suitable host animals include rabbits, mice, guinea pigs, rats, and fowl. Various adjuvants can be used to increase the immunological response, depending on the host species, including but not limited to Freund's (complete and incomplete adjuvant), adjuvant mineral gels such as aluminum hydroxide, surface active substances such as lysolecithin, pluronic polyols, polyanions, peptides, oil emulsions, keyhole limpet hemocyanin, dinitrophenol, BCG (bacille Calmette-Guerin) and Corynebacterium parvum. Such procedures result in the production of polyclonal antibodies, i.e., heterogeneous populations of antibody molecules derived from the sera of the immunized animals. Antibodies can be purified from blood obtained from the host animal, for example, by affinity chromatography methods in which the GR polypeptide antigen is immobilized on a resin.

The present invention also includes anti-GR monoclonal antibodies. Monoclonal antibodies (mAbs), which are homogeneous populations of antibodies to a particular antigen, can be prepared using GR polypeptides and standard hybridoma technology (see, e.g., Kohler et al., Nature, 256:495, 1975; Kohler et al., Eur. J. Immunol., 6:511, 1976; Kohler et al., Eur. J. Immunol., 6:292, 1976; Hammerling et al., In Monoclonal Antibodies and T Cell Hybridomas, Elsevier, NY, 1981; Ausubel et al., supra).

Typically, monoclonal antibodies are produced using any technique that provides for the production of antibody molecules by continuous cell lines in culture, such as those described in Kohler et al., Nature, 256:495, 1975, and U.S. Pat. No. 4,376,110; the human B-cell hybridoma technique (Kosbor et al., Immunology Today, 4:72, 1983; Cole et al., Proc. Natl. Acad. Sci. USA, 80:2026, 1983); and the EBV-hybridoma technique (Cole et al., Monoclonal Antibodies and Cancer Therapy, Alan R. Liss, Inc., pp. 77-96, 1983). Such antibodies can be of any immunoglobulin class including IgG, IgM, IgE, IgA, IgD, and any subclass thereof. The hybridomas producing the mAbs of this invention can be cultivated in vitro or in vivo.

Once produced, polyclonal or monoclonal antibodies can be tested for recognition, e.g., specific recognition, of GR in an immunoassay, such as a Western blot or immunoprecipitation analysis using standard techniques, e.g., as described in Ausubel et al., supra. Antibodies that specifically bind to a GR polypeptide, or conservative variants thereof, are useful in the invention. For example, such antibodies can be used in an immunoassay to detect a GR polypeptide in a sample, e.g., a tissue sample.

Alternatively or in addition, an antibody can be produced recombinantly, e.g., produced by phage display or by combinatorial methods as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

Anti-GR antibodies can be fully human antibodies (e.g., an antibody made in a mouse which has been genetically engineered to produce an antibody from a human immunoglobulin sequence), or non-human antibodies, e.g., rodent (mouse or rat), goat, primate (e.g., monkey), camel, donkey, porcine, or fowl antibodies.

An anti-GR antibody can be one in which the variable region, or a portion thereof, e.g., the CDRs, are generated in a non-human organism, e.g., a rat or mouse. The anti-GR polypeptide antibody can also be, for example, chimeric, CDR-grafted, or humanized antibodies. The anti-GR polypeptide antibody can also be generated in a non-human organism, e.g., a rat or mouse, and then modified, e.g., in the variable framework or constant region, to decrease antigenicity in a human.

Techniques developed for the production of “chimeric antibodies” (Morrison et al., Proc. Natl. Acad. Sci., 81:6851, 1984; Neuberger et al., Nature, 312:604, 1984; Takeda et al., Nature, 314:452, 1984) can be used to splice the genes from a mouse antibody molecule of appropriate antigen specificity together with genes from a human antibody molecule of appropriate biological activity. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,704,692 and 4,946,778) can be adapted to produce single chain antibodies against a GR polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments can include but are not limited to F(ab′)₂ fragments, which can be produced by pepsin digestion of the antibody molecule, and Fab fragments, which can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Polyclonal and monoclonal antibodies (or fragments thereof) that specifically bind to a GR polypeptide can be used, for example, to detect expression of GR or the presence of a GR allele, e.g., a truncated GR encoded by the allele and a GR with one or more of the mutations described herein, in a patient. For example, a GR polypeptide can be detected in conventional immunoassays of biological tissues or extracts. Examples of suitable assays include, without limitation, Western blotting, ELISAs, radioimmune assays, and the like.

### III. Diagnostic Methods

The disclosure also provides methods of identifying and/or categorizing a patient who is resistant, e.g., partially resistant, or hypersensitive to glucocorticoid treatment by determining whether the patient has a GR allele described herein, and making treatment decisions based on the type of the GR allele. Certain patients are resistant or less responsive to glucocorticoid treatment. Other patients are hypersensitive or hyper-responsive to glucocorticoid. It would be useful to identify these patients prior to the initiation of glucocorticoid treatment, since glucocorticoid can induce a number of undesirable side effects, e.g., immunosuppression, hyperglycemia, weight gain, muscle break down, glaucoma, and cataracts.

The term “patient” or “subject” is used throughout the specification to describe an animal, human or non-human, rodent or non-rodent, to whom treatment or diagnosis according to the methods of the present invention is provided. Veterinary and non-veterinary applications are contemplated. The term includes, but is not limited to, birds, reptiles, amphibians, and mammals, e.g., humans, other primates, pigs, rodents such as mice and rats, rabbits, guinea pigs, hamsters, cows, horses, cats, dogs, sheep and goats. Typical patients or subjects include humans, farm animals, and domestic pets such as cats and dogs.

Useful GR alleles to identify in patients in the methods described herein include those GR alleles encoding GR polypeptides that do not respond to or exhibit decreased responsiveness to glucocorticoid, e.g., GR polypeptides with decreased transactivation potential, e.g., GR alleles with mutations that decrease or inhibit GRs' ability to, e.g., bind to glucocorticoid, to homoderimerize, to translocate into the nucleus, to bind to a nucleic acid (e.g., to a glucocorticoid response element (GRE)), to bind to other transcription factors, or to induce or transactivate transcription of a gene. Patients with these kinds of GR alleles are expected to be resistant or less responsive to glucocorticoid treatment. If a patient is identified to have such a GR allele, the patient's treating physician can make a decision to administer an agent or a treatment other than glucocorticoid. For example, it is useful to identify patients with a GR allele with one or more mutations, e.g., deletions, insertions, or nucleic acid substitutions, that result in an early stop codon, and therefore a truncated GR polypeptide lacking at least a portion of the ligand binding domain. Other types of GR alleles to look for in patients are those that encode GR polypeptides with one or more amino acid substitutions in the ligand binding domain such that binding to glucocorticoid is abolished or decreased. For example, an amino acid substitution of an amino acid important for ligand binding (e.g., those subjected to posttranslational modification, e.g., phosphorylation) can lead to decreased affinity of the GR polypeptide for glucocorticoid.

Frame-shift mutations that substantially change the amino acid sequences of GR polypeptides are expected to result in non-functional GRs that do not respond to glucocorticoid. One or more amino acid substitutions, insertions, or deletions in the DNA binding domain of GRs, e.g., from nucleic acid changes, insertions, or deletions in exon 3, can result in GRs that do not bind to a GRE, and therefore do not transactivate genes that are normally induced by glucocorticoid. Patients who are resistant or less responsive to glucocorticoid treatment may have, for example, GR alleles that encode GRs with mutations in the transactivation domain, e.g., from nucleic acid changes, insertions, or deletions in exon 2, such that the GRs have decreased ability to interact with other transcription factors necessary for transactivation of genes induced by glucocorticoid.

Described herein are GR alleles encoding truncated GR polypeptides (e.g., from nucleotide changes, insertions, or deletions that result in a premature stop codon) that have decreased transactivation potential, see, e.g., Tables 2, 3A, 3B, 4 and 5. Also described herein are GRs with mutations in the DNA binding domain (e.g., nucleotide change G1379A, and corresponding amino acid change R460K) and/or the ligand binding domain (e.g., nucleotide change T2246C, and corresponding amino acid change F749G) that have decreased transactivation potential. In addition, described herein are GR alleles, e.g., with a 17-CAG repeat in exon 2, encoding GR polypeptides with decreased transactivation potential having a transactivation domain that is more homologous to the transactivation domain of a mouse GR than the transactivation domain of the reference human GR.

Other alleles to identify in patients include GR alleles encoding GR polypeptides that are hyper-responsive or hyper-sensitive to glucocorticoid, e.g., have increased or enhanced binding affinity and/or transactivation potential, e.g., GR alleles with mutations that increase the GRs' ability to, e.g., bind to glucocorticoid, to homoderimerize, to translocate into the nucleus, to bind to a nucleic acid (e.g., to a GRE), to bind to other transcription factors, or to induce or transactivate transcription of a gene. Patients with these kinds of GR alleles are expected to be more sensitive or responsive to glucocorticoid treatment. If a patient is identified to have such a GR allele, the patient's treating physician can make a decision, e.g., to administer lower dosages of glucocorticoid. For example, a patient who is more sensitive to glucocorticoid can have a GR allele that encodes a GR polypeptide with one or more amino acid substitutions, deletions, or insertions in the ligand binding domain such that the GR polypeptide has increased affinity for glucocorticoid. Mutations resulting in certain amino acid substitutions, deletions, or insertions in the DNA binding domain or the transactivation domain of a GR polypeptide could also increase the transactivation potential of the GR polypeptide. Described herein is a GR allele having a A2297G nucleotide change in exon 9α that encodes a GR polypeptide with a N766S amino acid substitution in the ligand binding domain, which exhibits significantly increased transactivation potential. The N766S mutations could potentially affect posttranslation modification of the GR polypeptide, and alters its transactivation potential.

Generally, a non-conservative amino acid substitution, in which an amino acid residue is replaced with an amino acid residue having a different kind of side chain, e.g., substitution of an amino acid with basic side chains for one with acidic side chains, can be more likely to result in a polypeptide with altered functions than conservative amino acid substitutions, e.g., substituting an amino acid with another with similar side chains.

The methods described herein are useful for patients with conditions, e.g., inflammatory disorders, that are routinely treated with glucocorticoids. Skilled practitioners can readily appreciate what disorders or conditions are routinely treated with glucocorticoids. As used herein, the term “inflammation” is used to describe the fundamental pathological process consisting of a dynamic complex of cytologic and histologic reactions that occur in the affected blood vessels and adjacent tissues in response to an injury or abnormal stimulation caused by a physical, chemical, or biologic agent, including the local reactions and resulting morphologic changes, the destruction or removal of the injurious material, and the responses that lead to repair and healing. The term includes various types of inflammation such as acute, allergic, alternative (degenerative), atrophic, catarrhal (most frequently in the respiratory tract), croupous, fibrinopurulent, fibrinous, immune, hyperplastic or proliferative, subacute, serous and serofibrinous. For example, inflammation can occur in the liver, heart, skin (e.g., dermatitis, inflammation due to bacterial, fungal, or viral infections and/or allergic or autoimmune reactions), spleen, brain, kidney (e.g., bacterial pyelonephritis, interstitial nephritis, and/or glomerulonephritis) and pulmonary tract, especially the lungs, and can be associated with sepsis or septic shock. A number of disorders and conditions, e.g., inflammatory disorders such as asthma, can cause or are associated with inflammation.

The methods described herein are applicable in a wide variety of clinical contexts. For example, the methods can be used for diagnosing patients in hospitals and outpatient clinics, as well as the Emergency Department. The methods can be carried out on-site or in an off-site laboratory.

Methods are known in the art to detect the presence or absence of a GR allele with one or more of the mutations described herein. For example, GR alleles can be detected by sequencing exons, introns, 5′ untranslated sequences, or 3′ untranslated sequences, by performing allele-specific hybridization, allele-specific restriction digests, mutation specific polymerase chain reactions (MSPCR), by single-stranded conformational polymorphism (SSCP) detection (Schafer et al. (1995) Nat. Biotechnol. 15:33-39), denaturing high performance liquid chromatography (DHPLC, Underhill et al. (1997) Genome Res. 7:996-1005), infrared matrix-assisted laser desorption/ionization (IR-MALDI) mass spectrometry (WO 99/57318), and combinations of such methods.

Genomic DNA can be used in the analysis of GR alleles. Genomic DNA typically is extracted from a biological sample such as a peripheral blood sample, but also can be extracted from other biological samples, including tissues (e.g., mucosal scrapings of the lining of the mouth or from renal or hepatic tissue). Standard methods can be used to extract genomic DNA from a blood or tissue sample, including, for example, phenol extraction. Alternatively, genomic DNA can be extracted with kits such as the Qiagen DNeasy Kit, the QIAamp® Tissue Kit (Qiagen, Valencia, Calif.), Wizard® Genomic DNA purification kit (Promega, Madison, Wis.) and the A.S.A.P.™ Genomic DNA isolation kit (Boehringer Mannheim, Indianapolis, Ind.).

cDNA can also be used in the analysis of GR alleles. Using conventional methods, cDNA can be synthesized from the mRNA fraction of the total RNA isolated from a biological sample.

An amplification step can be performed before proceeding with the detection method. For example, exons and/or introns of a GR gene can be amplified and then directly sequenced. Primers useful for amplification of portions of a GR gene are described above, e.g., SEQ ID NOs: 3-61. High throughput automated (e.g., capillary or microchip based) sequencing apparati can be used. Nucleic acid analysis include sequencing with a pyrophosphate DNA sequencer (454 Life Sciences, New Haven, Conn.; see U.S. Pat. Pub. No. 20050130173) or optical sequencing (see, e.g., U.S. Pat. Pub. Nos. 20060024711, 20060136144, and 20060012793).

Mass spectroscopy (e.g., MALDI-TOF mass spectroscopy) can be used to detect nucleic acid mutations. In some cases (e.g., the MassEXTEND™ assay, SEQUENOM, Inc.), selected nucleotide mixtures, missing at least one dNTP and including a single ddNTP is used to extend a primer that hybridizes near a mutation. The nucleotide mixture is selected so that the extension products between the different polymorphisms at the site create the greatest difference in molecular size. The extension reaction is placed on a plate for mass spectroscopy analysis.

Fluorescence based detection can also be used to detect nucleic acid mutations. For example, different terminator ddNTPs can be labeled with different fluorescent dyes. A primer can be annealed near or immediately adjacent to a mutation, and the nucleotide at the mutation site can be detected by the type (e.g., “color”) of the fluorescent dye that is incorporated.

Hybridization to microarrays can also be used to detect mutations. For example, a set of different oligonucleotides, with the mutant nucleotide at varying positions with the oligonucleotides can be positioned on a nucleic acid array. The extent of hybridization as a function of position and hybridization to oligonucleotides specific for the other allele can be used to determine whether a particular mutation is present. See, e.g., U.S. Pat. No. 6,066,454.

Hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other.

Allele specific hybridization also can be used to detect GR alleles, including complete haplotypes of a mammal. See, Stoneking et al. (1991) Am. J. Hum. Genet. 48:370-382; and Prince et al. (2001) Genome Res. 11:152-162. For example, samples of DNA or RNA from one or more patient can be amplified using pairs of primers and the resulting amplification products can be immobilized on a substrate (e.g., in discrete regions). Hybridization conditions can be selected such that a nucleic acid probe can specifically bind to the sequence of interest, e.g., the GR nucleic acid containing a particular GR allelic nucleotide sequence. Such hybridizations typically are performed under high stringency, as some allelic nucleotide sequences include only a single nucleotide difference. High stringency conditions can include, for example, the use of low ionic strength solutions and high temperatures for washing. For example, nucleic acid molecules can be hybridized at 42° C. in 2×SSC (0.3M NaCl/0.03M sodium citrate/0.1% sodium dodecyl sulfate (SDS) and washed in 0.1×SSC (0.015M NaCl/0.0015M sodium citrate), 0.1% SDS at 65° C. Hybridization conditions can be adjusted to account for unique features of the nucleic acid molecule, including length and sequence composition. Probes can be labeled (e.g., fluorescently) to facilitate detection. In some cases, one of the primers used in the amplification reaction is biotinylated (e.g., 5′ end of reverse primer) and the resulting biotinylated amplification product is immobilized on an avidin or streptavidin coated substrate.

Allele-specific restriction digests can be performed in the following manner. For GR allelic nucleotide sequences that introduce a restriction site, restriction digest with the particular restriction enzyme can differentiate the alleles. For GR allelic nucleotide sequences that do not alter a common restriction site, mutagenic primers can be designed that introduce a restriction site when the variant allele is present or when the wild type allele is present. A portion of a GR nucleic acid can be amplified using the mutagenic primer and a wild type primer, followed by digest with the appropriate restriction endonuclease.

Certain alleles, such as those with insertions or deletions of one or more nucleotides, can change the size of the DNA fragment encompassing the allele. The insertion or deletion of nucleotides can be assessed by amplifying the region encompassing the allele and determining the size of the amplified products in comparison with size standards. For example, a region of a GR nucleic acid can be amplified using a primer set from either side of the allele. One of the primers can be labeled, for example, with a fluorescent moiety, to facilitate sizing. The amplified products can be electrophoresed through acrylamide gels with a set of size standards that are labeled with a fluorescent moiety that differs from the primer.

PCR conditions and primers can be developed that amplify a product only when a particular allelic nucleic acid sequence is present or only when the wild type nucleic acid sequence is present (MSPCR or allele-specific PCR). For example, DNA from a patient and a control can be amplified separately using either a wild type GR primer or a primer specific for a GR allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction. Patient samples containing solely the wild type GR nucleic acid sequence would have amplification products only in the reaction using the wild type primer. Similarly, patient samples containing solely an GR allele would have amplification products only in the reaction using the allele-specific primer. Allele-specific PCR also can be performed using allele-specific primers that introduce priming sites for two universal energy-transfer-labeled primers (e.g., one primer labeled with a green dye such as fluoroscein and one primer labeled with a red dye such as sulforhodamine). Amplification products can be analyzed for green and red fluorescence in a plate reader. See, Myakishev et al. (2001) Genome 11:163-169.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

Alternatively or in addition, the presence of a GR allele in a patient can be detected by analyzing the GR polypeptide encoded by the allele. For example, an allele that gives rise to a truncated GR polypeptide can be detected by using an anti-GR antibody that recognizes the truncated GR polypeptide, e.g., by Western blotting. Useful anti-GR antibodies and methods of making thereof are described above.

An exemplary method for determining whether a patient has a GR allele described herein can include drawing a sample, e.g., about 1 ml, of blood from the patient, then isolating genomic DNA from the blood sample using any methods described herein or known the art. A specific region, e.g., exon 2, or exon 9α, that contains one or more mutations of interest of the GR gene can be amplified by PCR using a set of primers, e.g., the primers listed in Table 1. The amplified PCR product can be purified, cloned into plasmids, and sequenced to determine whether the patient has a GR allele with one or more mutations described herein. For example, primers 9α-1F (SEQ ID NO: 34) and 9α-2C (SEQ ID NO: 37) can be used to amplify exon 9α of the GR gene of the patient to determine whether the patient has a GR allele with a A2297G mutation in exon 9α. A patient with this GR allele is expected to be hypersensitive to glucocorticoid treatment. In another example, primers 2-2D (SEQ ID NO: 60) and 2-2E (SEQ ID NO: 61) can be used to amplify exon 2 of the GR gene of the patient to determine whether the patient has a GR allele with a 17-CAG repeat in exon 2, e.g., a “mouse-like” GR allele. This patient is expected to be less responsive to glucocorticoid treatment. Primers 4-1B (SEQ ID NO: 13) and 4-2B (SEQ ID NO; 15) can be used to amplify exon 4 of the GR gene to determine whether there is a mutation in the DNA binding domain of the GR. Those skilled in the art can appreciate that any combination of a forward primer and a reverse primer, e.g., those primers listed in Table 1, for a particular exon of the GR gene can be used to amplify the exon to determine whether that exon contains one or more mutations of interest.

### IV. Screening Methods

Described herein are methods of identifying compounds (e.g., glucocorticoid or non-glucocorticoid compounds) that are effective for activating specific GR alleles or mutants. Also provided are methods of identifying novel compounds to treat patients who are resistant to glucocorticoids.

Glucocorticoids

A number of glucocorticoids, e.g., synthetic glucocorticoids, are available for treating a variety of inflammatory disorders, autoimmune disorders, allergic reactions, and other conditions that need anti-inflammatory or immunosuppressive treatments. Exemplary glucocorticoids include, but are not limited to, betamethasone, budesonide, cortisone acetate, dexamethasone, fludrocortisones acetate, hydrocortisone, hydrocortisone sodium succinate, methylprednisolone, methylprednisolone acetate, methylprednisolone sodium succinate, prednisolone acetate, prednisolone sodium phosphate, prednisone, triamcinolone, triamcinolone acetonide, triamcinolone diacetate, and triamcinolone hexacetonide.

Non-Glucocorticoid Anti-Inflammatory Agents

Examples of non-glucocorticoid anti-inflammatory agents include, but are not limited to NSAIDS (non-steroidal anti-inflammatory drugs) such as acetaminophen; salicylates, e.g., aspirin, methyl salicylate and diflunisal; arylalkanoic acids, e.g., diclofenac, indomethacin, and sulindac; 2-arylpropionic acids, e.g., ibuprofen, ketoprofen, naproxen, carprofen, fenoprofen, and ketorolac; N-arylanthranilic acids, e.g., mefenamic acid; oxicams, such as piroxicam and meloxicam; sulfonanilides, e.g., nimesulide; and COX-2 inhibitors, e.g., celecoxib, rofecoxib, valdecoxib, parecoxib, and etoricoxib.

Libraries of Test Compounds

In some screens disclosed herein, libraries of test compounds are used. As used herein, a “test compound” can be any chemical compound, for example, a macromolecule (e.g., a polypeptide, a protein complex, glycoprotein, polysaccharide, or a nucleic acid) or a small molecule (e.g., an amino acid, a nucleotide, or an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids, retro-peptides, inverso peptides, and retro-inverso peptides), amino acids, amino acid analogs, polynucleotides, polynucleotide analogs, nucleotides, nucleotide analogs, and organic or inorganic compounds, e.g., heteroorganic or organometallic compounds.

Test compounds can also be known glucocorticoids that are altered in some systematic way to generate large numbers of test compounds. In addition, test compounds can be known non-glucocorticoid anti-inflammatory agents that are either in their native state or altered in a systematic way to generate large numbers of test compounds.

Test compounds can be screened individually or in parallel. An example of parallel screening is a high throughput drug screen of large libraries of chemicals. Such libraries of candidate compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. Alternatively, prior experimentation and anecdotal evidence can suggest a class or category of compounds of enhanced potential. A library can be designed and synthesized to cover such a class of chemicals.

The synthesis of combinatorial libraries is well known in the art and has been reviewed (see, e.g., Gordon et al., J. Med. Chem., 37:1385-1401 (1994); Hobbes et al., Acc. Chem. Res., 29:114 (1996); Armstrong, et al., Acc. Chem. Res., (1996) 29:123; Ellman, Acc. Chem. Res., (1996) 29:132; Gordon et al., Acc. Chem. Res., 29:144 (1996); Lowe, Chem. Soc. Rev., 309 (1995); Blondelle et al., Trends Anal. Chem., 14:83 (1995); Chen et al., J. Am. Chem. Soc., 116:2661 (1994); U.S. Pat. Nos. 5,359,115, 5,362,899, and 5,288,514; PCT Publication Nos. WO92/10092, WO93/09668, WO91/07087, WO93/20242, and WO94/08051).

Libraries of compounds, e.g., glucocorticoids and/or non-glucocorticoid anti-inflammatory compounds or analogs thereof, can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky “Principles of Peptide Synthesis,” 2nd edition, Springer-Verlag, Berlin (1993)). To each aliquot of beads is added a solution of a different activated amino acid, and the reactions are allowed to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy can result in a library of peptides, e.g., modulators, which can be used to prepare a library of test compounds of the invention. In another illustrative synthesis,a “diversomer library” is created by the method of Hobbs DeWitt et al. (Proc. Natl. Acad. Sci. U.S.A., 90:6909 (1993)). Other synthesis methods, including the “tea-bag” technique of Houghten (see, e.g., Houghten et al., Nature, 354:84-86 (1991)) can also be used to synthesize libraries of compounds according to the subject invention.

Libraries of compounds can be screened to determine whether any members of the library can modulate anti-inflammatory responses in cells or animal models with a GR allele that confers glucocorticoid resistance, and, if so, to identify the compound. Methods of screening combinatorial libraries have been described (see, e.g., Gordon et al., J Med. Chem., supra). Exemplary assays useful for screening libraries of test compounds are described above.

Screening Methods

In some screens, known glucocorticoids, e.g., synthetic glucocorticoid, can be tested to determine whether and which glucocorticoid is effective for activating the GR polypeptide encoded by a specific GR allele described herein, e.g., a GR polypeptide that is still functional, but may be less responsive to certain glucocorticoids. An exemplary screen can be carried out as follows. The GR nucleic acid sequence encoding the GR polypeptide of interest can be cloned into a first vector (e.g., pcDNA4, Invitrogen, Carlsbad, Calif.). A second vector can be constructed to contain a reporter gene, e.g., the luciferase gene, under the control of a GRE. These two vectors can be co-transfected into a cell, e.g., a human embryonic kidney cell (HEK 293). Skilled practitioners will recognize that a number of cell types can be used in the screen. The cell will then express the GR polypeptide encoded by the GR nucleic acid sequence on the first vector. Different glucocorticoids can be administered to the cell to determine whether and how strongly any of them can affect or alter the transcription activation potential of the GR polypeptide as compared to controls, e.g., cell grown in media with no glucocorticoid or only a basal level of glucocortioid provided by, e.g., fetal bovine serum. If the GR polypeptide is responsive to a glucocorticoid, it will bind to the GRE and transactivate the transcription of the reporter gene. The product of the reporter gene, e.g., luciferase, can then be detected and quantified using conventional methods. For example, when a luciferin substrate is added, luciferase will produce luminescence that can be detected and quantified. The amount of bioluminescence produced is therefore proportional to the amount of luciferase produced and, consequently, to the activation potential of the GR protein expressed in the cell. The results of cell-based assays can be further confirmed by, for example, testing glucocorticoids in animal models containing the GR allele of interest.

Also described herein are methods of identifying novel compounds for treating inflammation. The compounds identified are useful for treating patients who are resistant to glucocorticoids. For example, cells or animal models expressing a GR polypeptide that is not responsive to glucocorticoid can be treated with test compounds to determine whether any test compound can induce an anti-inflammatory response, e.g., a response that is normally induced by glucocorticoid in a cell expressing a wild-type GR. Anti-inflammatory responses can include, but are not limited to, expression of an anti-inflammatory gene, e.g., interleukin-11. Skilled practitioners can readily appreciate that expression of a number of anti-inflammatory genes and other anti-inflammatory responses can be monitored in the screening assays provided herein.

### V. Kits

Also provided herein are kits for detecting the presence of a GR allele in a cell, a sample or a patient, for example, for the screening and diagnostic methods described herein.

Kits can include primers described herein, which can be used to detect a GR allele. Kits may include, e.g., instructional material on how to use the primers to detect a GR allele. The informational material can be descriptive, instructional, marketing or other material that relates to the screening and diagnostic methods described herein.

For example, a kit for determining whether a patient is resistant to glucocorticoid treatment can include one or more pairs of forward and reverse primers, e.g., SEQ ID NO: 34 and SEQ ID NO: 37, SEQ ID NO: 34 and SEQ ID NO: 53, and SEQ ID NO: 30 and SEQ ID NO: 54, for amplifying exon 9α of the GR gene to determine whether there are mutations, e.g., a deletion of nucleotides 2201-2204, that result in at least a partial deletion of the ligand binding domain of the GR. Such a kit can also be used to detect whether there are one or more nucleic acid changes in exon 9α that result in amino acid substitutions in the ligand binding domain of the GR such that the GR exhibits decreased transactivation potential, e.g., resistance to glucocorticoid.

Alternatively or in addition to primers for exon 9α, a kit for determining whether a patient is less responsive to glucocorticoid treatment can include one or more pairs of primers for exon 2 of the GR gene to detect one ore more mutations in exon 2, e.g., resulting in mutations in the transactivation domain of the GR, that result in a GR with decreased transactivation potential. For example, one or more pairs of primers, e.g., SEQ ID NO: 4 and SEQ ID NO: 5, SEQ ID NO: 4 and SEQ ID NO: 6, SEQ ID NO: 46 and SEQ ID NO: 8, and SEQ ID NO: 45 and SEQ ID NO: 7, can be used to amplify exon 2. Other pairs primers, e.g., SEQ ID NO: 4 and SEQ ID NO: 60, can also be used to amplify exon 2 to determine whether a patient has a GR allele containing a 17-CAG repeat in exon 2. A patient having such a GR allele is expected to be less responsive to glucocorticoid treatment.

A kit for determining whether a patient is resistant to glucocorticoid treatment can also include one or more pairs of primers for exon 4 of the GR gene to detect mutations in exon 4, e.g., resulting in mutations in the DNA binding domain of GR, e.g., a G1379A mutation, that result in a GR with decreased transactivation potential. Useful pairs of primers for exon 4 can include those having the sequences of SEQ ID NO: 13 and SEQ ID NO: 15, and SEQ ID NO: 12 and SEQ ID NO: 14.

A kit for determining whether a patient is hypersensitive to glucocorticoid can include one or more pairs of forward and reverse primers, e.g., SEQ ID NO: 34 and SEQ ID NO: 37, SEQ ID NO: 34 and SEQ ID NO: 53, and SEQ ID NO: 30 and SEQ ID NO: 54, for amplifying exon 9α of the GR gene to determine whether there are mutations, e.g., a A2297G mutation, associated with glucocorticoid hyper-sensitivity. Such a kit can also include, alternatively or additionally, one or more pairs of primers for one or more other exons, e.g., exon 2 and exon 4, to detect nucleic acid changes, deletion and insertions that result in mutation in the transactivation domain or DNA binding domain of the GR, such that the GR has increased transactivation potential, e.g., enhanced responsiveness to glucocorticoid. Patients having these alleles are expected to by hypersensitive to glucocorticoid treatment.

In another example, a kit can include one or more pairs of forward and reverse primers, e.g., those primers described herein, for each of exons 2, 3, 4, 5, 6, 7, 8 9α and 9β of the GR gene, which would allow detection of all mutations of interest present in the entire GR gene, e.g., mutations in all of the exons, e.g., mutations in the transactivation domain, the DNA binding domain, and the ligand binding domain. The kit can be used to categorize a patient as to his or her overall sensitivity to glucocorticoid treatment by identifying all relevant GR mutations.

The kits described herein can be used to carry out the diagnostic methods described herein using the primers provided in the kits. For example, the kit can be used to PCR amplify specific GR regions from genomic DNA isolated from a blood sample obtained from a patient. The amplified PCR products can be cloned into plasmids and sequenced to determine whether the patient has a GR allele with one or more of the mutations described herein.

Kits may include GR polypeptides described herein, e.g., a truncated polypeptide, described above, for example, for use in screening methods described herein. In some instances, the kits can include instructional material on how to use the GR polypeptides for these screening methods. The informational material can be descriptive, instructional, marketing or other material that relates to the screening methods described herein.

The informational material of the kit is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about GR or the primers and/or their use in the screening, diagnostic and therapeutic methods described herein. Of course, the informational material can also be provided in any combination of formats.

In addition to GR polypeptides and/or primers, a kit can include other ingredients, such as a solvent or buffer, and/or other agents for practicing the screening, diagnostic and therapeutic methods described herein. Such a kit can include instructions for using GR polypeptides or primers together with the other ingredients.

GR polypeptides can be provided in any form, e.g., liquid, dried or lyophilized form. These can be provided in, e.g., substantially pure and/or sterile form. When GR polypeptides are provided in a liquid solution, the liquid solution can be an aqueous solution, e.g., a sterile aqueous solution.

A kit can include one or more containers for the composition containing a GR polypeptide or a primer. The kit can include separate containers, dividers or compartments for the composition and informational material. For example, the composition can be contained in a bottle, vial, or syringe, and the informational material can be contained in a plastic sleeve or packet. The separate elements of the kit can be contained within a single, undivided container. For example, the composition can be contained in a bottle, vial or syringe that has attached thereto the informational material in the form of a label. The kit may include a plurality (e.g., a pack) of individual containers, each containing one composition including a GR polypeptide or a primer. For example, the kit can include a plurality of syringes, ampoules, foil packets, or blister packs, each containing a composition including a GR polypeptide or primer. The containers of the kits can be air tight and/or waterproof.

### VII. Transgenic Animals

The present invention also features transgenic animals that express certain GR polypeptides. Such animals represent model systems for the study of disorders that are caused by or exacerbated by mutations in GR and for the development of therapeutic agents treating these disorders.

Transgenic animals can be, for example, farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats). A “transgene” is exogenous DNA that is integrated into the genome of a cell from which a transgenic animal develops and which remains in the genome of the mature animal, thereby directing the expression of an encoded gene product in one or more cell types or tissues of the transgenic animal. A transgene can also be created to remove or disrupt the expression of an endogenous gene.

Any technique known in the art can be modified as described herein to introduce a GR transgene into animals to produce the founder lines of transgenic animals. Such GR transgenes would encode for a mutated GR polypeptide, e.g., a GR polypeptide described herein. Such techniques include, but are not limited to, pronuclear microinjection (U.S. Pat. No. 4,873,191); retrovirus mediated gene transfer into germ lines (Van der Putten et al., Proc. Natl. Acad. Sci. USA 82:6148, 1985); gene targeting into embryonic stem cells (Thompson et al., Cell 56:313, 1989); and electroporation of embryos (Lo, Mol. Cell. Biol. 3:1803, 1983). Especially useful are the methods described in Yang et al. (Proc. Natl. Acad. Sci. USA 94:3004-3009, 1997). Construction of a transgenic animal that overexpresses a GR transgene is described below in the Examples section.

The present invention provides for transgenic animals that carry a GR transgene in all their cells, as well as animals that carry the transgene in some, but not all, of their cells. That is, the invention provides for mosaic animals. The transgene can be integrated as a single transgene or in concatamers, e.g., head-to-head tandems or head-to-tail tandems. The transgene can also be selectively introduced into and activated in a particular cell type (Lasko et al., Proc. Natl. Acad. Sci. USA 89:6232, 1992). The regulatory sequences required for such a cell-type specific activation will depend upon the particular cell type of interest, and will be apparent to those of skill in the art.

When it is desired that the GR transgene be integrated into the chromosomal site of the endogenous GR gene, gene targeting is preferred. Briefly, when such a technique is to be used, vectors containing some nucleotide sequences homologous to an endogenous GR gene are designed for the purpose of integrating, via homologous recombination with chromosomal sequences, into and disrupting the function of the nucleotide sequence of the endogenous gene. The transgene also can be selectively introduced into a particular cell type, thus inactivating the endogenous GR gene in only that cell type (Gu et al., Science 265:103, 1984). The regulatory sequences required for such a cell-type specific inactivation will depend upon the particular cell type of interest, and will be apparent to those of skill in the art. These techniques are useful for preparing “knock outs” having no functional GR gene.

Once transgenic animals have been generated, the expression of the recombinant GR gene can be assayed utilizing standard techniques. Initial screening may be accomplished by Southern blot analysis or PCR techniques to determine whether integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and RT-PCR. Samples of GR gene-expressing tissue can also be evaluated immunocytochemically using antibodies specific for wild type GR or the GR transgene product.

For a review of techniques that can be used to generate and assess transgenic animals, skilled artisans can consult Gordon (Intl. Rev. Cytol. 115:171-229, 1989), and may obtain additional guidance from, for example: Hogan et al. Manipulating the Mouse Embryo, Cold Spring Harbor Press, Cold Spring Harbor, N.Y., 1986); Krimpenfort et al. (Bio/Technology 9:86, 1991), Palmiter et al. (Cell 41:343, 1985), Kraemer et al. (Genetic Manipulation of the Early Mammalian Embryo, Cold Spring Harbor Press, Cold Spring Harbor, N.Y., 1985), Hammer et al. (Nature 315:680, 1985), Purcel et al. (Science, 244:1281, 1986), Wagner et al. (U.S. Pat. No. 5,175,385), and Krimpenfort et al. (U.S. Pat. No. 5,175,384).

### EXAMPLES

The following examples are illustrative and not limiting.

### Example 1

### Analysis of GR Alleles in Human Subjects

In this example, samples from 97 human subjects were collected and their GR sequences were analyzed.

Samples of blood were obtained by venipuncture from the human subjects. Total RNA and/or genomic DNA were purified from the mononuclear cells in the blood.

For subjects 1-15, genomic DNA was isolated and the human glucocorticoid receptor (GR)-α was sequenced by examining each exon individually, except for exon 1, which does not contain sequences that are translated into protein. Using primer sets designed specifically for each exon based on the reference sequence (SEQ ID NO: 1), the exons were amplified by polymerase chain reaction (PCR). These PCR products were purified, then cloned into the pGEM®-T Easy vector (Promega, Madison, Wis.). Vectors from multiple clones for each subject were sequenced. The sequences were then analyzed using sequencing analysis software. Since each subject may have two different alleles for the human GR-α (inherited from each parent), there may be more than one sequence available for each subject.

Table 2 summarizes the nucleotide insertions, deletions, and point mutations found in the GR gene of subjects 1-15 as compared to the reference GR sequence (SEQ ID NO: 1), and also list changes to the amino acids resulting from these nucleotide changes. Mutations are abbreviated as (reference nucleotide) (location of change) (altered nucleotide) for the nucleotide point mutations or (reference amino acid) (location of change) (altered amino acid) for the amino acid changes. A “+” or “−” indicates an insertion or deletion of a nucleotide/amino acid at a particular position. For example, in subject 7, a T916C mutation was found in exon 2 of the GR gene in clone 5. This mutation leads to a F306L amino acid substitution in the GR polypeptide. In another example, in clone 3 from human subject 2, there is a deletion of 54 nucleotides, including deletion of nucleotides 1378-1382, 1386-1389, 1391-1418, 1421-1425, 1428-1429, and 1431-1441. These deletions result in three amino acid substitutions and the deletion of residues 464 to 481.

For all subjects except subjects 88 and 90, cDNA was synthesized from the mRNA fraction of the total RNA isolated from the samples. Two primer sets were used, one encompassing the segment of exons 2 to 3 and the other from exons 3 to 9α, to amplify the human GR-α by PCR from the cDNA. The segments were purified, then cloned into pGEM®-T Easy vector. Vectors from multiple clones for each subject were sequenced. The sequences were then analyzed by sequencing analysis software. The presumed GR-α sequence was then assembled in silico from the exon 2-3 and exon 3-9α sequences. Since each subject may have inherited different alleles from each parent, there may be several sequences listed for each subject, a result of the assembly of the separate sections of the gene that were sequenced.

Table 3A summarizes the mutations found in exons 2 and 3 of the GR sequences from the 97 subjects based on the cDNA data. Table 3B summarizes the mutations found in the cDNA fragment encompassing exons 3 to 9α from the 97 subjects. Mutations are abbreviated as (reference nucleotide) (location of change) (altered nucleotide) for the point mutations or (reference amino acid) (location of change) (altered amino acid) for the changes to amino acid sequences. A “+” or “−” indicates an insertion or deletion of a nucleotide/amino acid at a particular position. Each of Tables 3A and 3B contains multiple sections: one listing clones with nucleotide changes, insertions or deletions that result in amino acid changes; one listing clones with nucleotide changes, insertions or deletions that result in alternative stop such that the GRs are truncated or extended; and one listing clones with sequences that share homology with a mouse GR.

From the cDNA analysis, a few subjects (13, 79, 81, 83) were found to have a stretch of 17 CAG repeats in exon 2 of their GR gene that closely resembles a region in exon 1 of the mouse GR (mGR) of the C57BL/6J strain (see Table 3A). The 17-CAG repeat encodes a 17-glutamine repeat in the transactivation domain of the mGR and the GRs of these 4 subjects. These “mouse-like” human GR sequences were confirmed by analyzing the genomic DNA data obtained from subjects 79, 81, and 83. Table 4 summarizes the genomic DNA data. FIG. 3A shows a sequence alignment between the C57BL/6J mGR nucleic acid sequence (Genbank Accession No. NM_{—}008173; SEQ ID NO: 62), the reference human GR nucleic acid sequence (SEQ ID NO: 1) and the GC nucleic acid sequences from subjects 13 (SEQ ID NO: 63), 79 (SEQ ID NO: 64), 81 (SEQ ID NO: 65) and 83 (SEQ ID NO: 66). FIG. 3B shows a sequence alignment between the C57BL/6J mGR amino sequence (SEQ ID NO: 67; from translation of SEQ ID NO: 62), the reference human GR amino sequence (SEQ ID NO: 2) and the GR amino acid sequences from subjects 13 (SEQ ID NO: 68), 79 (SEQ ID NO: 69), 81 (SEQ ID NO: 69) and 83 (SEQ ID NO: 70).

### Example 2

### Analysis of Transactivation Potential of GR Alleles in Humans

In this example, the transactivation potential of polypeptides encoded by some GR alleles identified in Example 1 was analyzed.

Blood samples were obtained from the volunteer subjects after protocol approval by the Institutinal Review Board. The buffy coat was collected for RNA isolation. Reverse transcription and polymerase chain reactions (PCR) were performed on the RNA to amplify the GR gene from each subject using specially designed primers, as described above. The entire GR coding sequences were amplified in two separate sections, exons 2-3 and exons 3-9α, and these were recombined to generate the full-length GR expression plasmid using a pcDNA-HisMax expression vector (Invitrogen).

Table 5 summarizes the mutations present in these recombinant GRs. The left side of Table 5 (under the heading Mutations Present in Reconstructed hGR Allele) shows the actual sequences of the GR alleles cloned. The right side of Table 5 (Expected Mutations) shows the sequences expected from the in silico analysis of the cDNA GR alleles originally analyzed (see Tables 3A and 3B). For some alleles, there are differences between the sequences of the reconstructed alleles and the expected sequences from the in silico analysis, as explained in the legend of Table 5. All GR alleles described in the functional analysis refer to the alleles containing the mutations listed in the reconstructed sequences, not the expected in silico sequences. Each allele is identified by (subject #) (clone # of exons 2-3) (clone # of exons 3-9α). For example, 22-14 indicates subject 22, with the exon 2-3 sequence from clone 1 and the exon 3-9α sequence from clone 4 (see Tables 3A and 3B).

For the GR allele from subject 13, an initial analysis revealed that the exon 2-3 portion of the allele closely resembles the reference mGR sequence, including the 17 CAG repeats found in the transactivation domain or the reference mGR, but not in the reference human GR (SEQ ID NO: 1). The matching mouse-like hGR 3-9α segment for this subject has not been identified; all the isolated exon 3-9α clones for this subject resemble the reference human GR. Therefore, the reconstructed full-length GR allele from subject 13 (13-2a-11-6) contains the cloned mouse-like exon 2-3 combined with the human-like exon 3-9α segment. FIG. 4 is a schematic representation of the mutations in these recombinant GR allelic sequences and their corresponding polypeptides.

The reconstructed GR allele expression plasmids described above were co-transfected with a GRE (glucocorticoid response element)-Luciferase reporter plasmid (Stratagene) using Fugene HD/6 (Roche) into HEK (human embryonic kidney) 293 cells, grown in Dulbecco's Modified Eagle Medium with 10% fetal bovine serum and 1% Penicillin/Streptomycin (Invitrogen). All transfections were done in triplicate. The cells were then lysed and assayed for luciferase activity. If the GR polypeptides expressed from the GR expression plasmids were functional, they would bind to the GRE and transactivate the transcription of the luciferase reporter gene. Luciferase luminescence was measured on the Perkin-Elmer MicroBeta TriLux machine and read in triplicate to ensure stability. The differences in luminescence were graphed and analyzed.

Six of the reconstructed GR alleles (22-23, 25-34, 66-15, 66-65, 50-51 and 27-51) contain nucleic acid sequence changes that should result in an early amino acid termination. Three other alleles (66-12, 66-62, and 66-63) also contain significant nucleic acid changes that result in amino acid alterations. FIG. 5 shows the transactivation potential of GR polypeptides encoded by these nine alleles. FIG. 5 shows fold change in luceriferase activity relative to the luceriferase activity induced by the reference human GR polypeptide (SEQ ID NO: 2). The six early termination (i.e., truncated) GR polypeptides exhibited fold changes of approximately 0.1 (i.e., decreased activity, approximately a tenth of the activity induced by the reference GR). Allele 66-63, which encodes a full-length polypeptide that contains amino acid substitutions, also exhibited very low level of luciferase activity. In contrast, allele 66-62, which also encodes a full-length GR with amino acid substitutions, exhibited a fold increase of 5.5, indicating a significant increase in transactivation potential. GR 66-12 had lower transactivation potential as compared to GR 66-62, but exhibited two and a half times higher activity (fold change 2.5) as compared to the reference GR. This experiment was repeated six times.

In summary, all six truncated GR polypeptides exhibited reduced transactivation potential as compared to the reference GR, e.g., are resistant to glucocorticoid. GR 66-63, which is a full-length GR that contains amino acid substitutions, like the truncated GRs, exhibited reduced transactivation potential. GRs 66-12 and 66-62, which are full-length GRs that contain amino acid substitutions, exhibited higher transactivation potential, e.g., are hypersensitive to glucocorticoid.

GR 66-62 and GR 66-63 are both full-length GRs with single amino acid substitutions, but they exhibited opposite transactivation potentials. These two GR alleles share two nucleotide changes, A214G and T962C, suggesting that these changes are unlikely to be responsible for the altered transactivation potentials. However, the only other mutation found in allele 66-62, an A2297G change, is not present in allele 66-63 (see FIG. 3 and Table 5). The same A2297G change is also present in allele 66-12, which also encodes a GR that exhibited increased transactivation potential. The A2297G change results in an N766S amino acid substitution in the ligand binding domain of the GR. The N766S amino acid change is expected to be responsible for the hyper-responsiveness of GRs 66-62 and 66-63 to glucocorticoid.

Allele 66-63, in addition to A214G and T962C, contains two other nucleotide changes, G1379A and T2246C. These two changes are not present in alleles 66-12 or 66-62, suggesting that one or both of these two nucleotide changes are responsible for the reduced transactivation potential exhibited by GR encoded by allele 66-63. A G1379A change causes a R460K amino acid substitution in the DNA binding domain. A T2246C nucleotide change results in a F749G amino acid substitution in the ligand binding domain.

As described above, four subjects in this study have regions in their GR polypeptides that are similar to the N-terminus transactivation domain of the C57BL/6J mGR. It is anticipated that the corresponding C-terminus region (exons 3-9α) of a “mouse-like” human GR polypeptide would be identified in those human subjects. The transactivation potential of a reconstructed mouse-like GR polypeptide from subject 13 (13-2a-11-6) is shown in FIG. 6. In comparison to the reference C57BL/6J mGR (W11) and the reference human GR, the transactivation potential of GR 13-2a-11-6 is significantly lower, though is slightly higher than the transactivation potential of the negative control (W8; a non-functional GR) and the early termination GR 27-51. The transactivation potential of GR 13-2a-11-6 is comparable to that of the K₉ mGR, which has an 8-CAG repeat (allele 13-2a-11-6 has a 17-CAG repeat). The results were repeated in a second assay.

TABLE 2
Human Subjects 1-15 (genomic DNA data) - List of GR Clones and Mutations

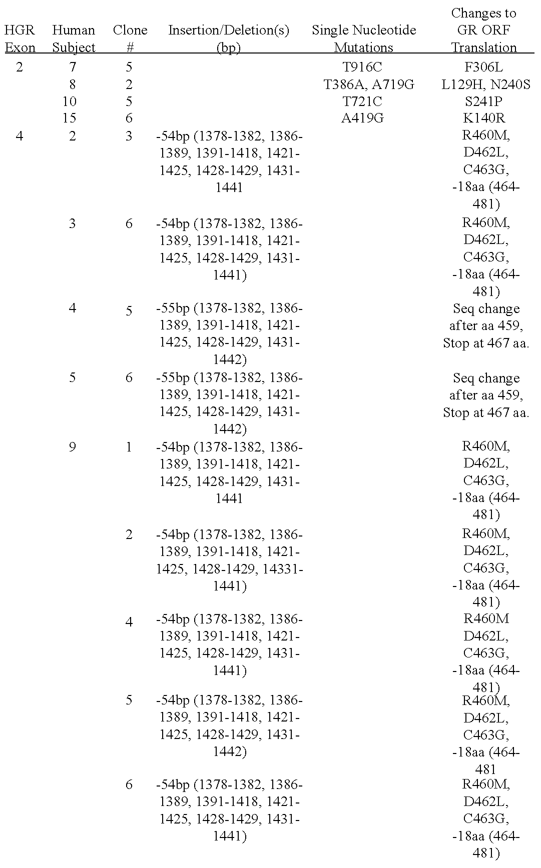

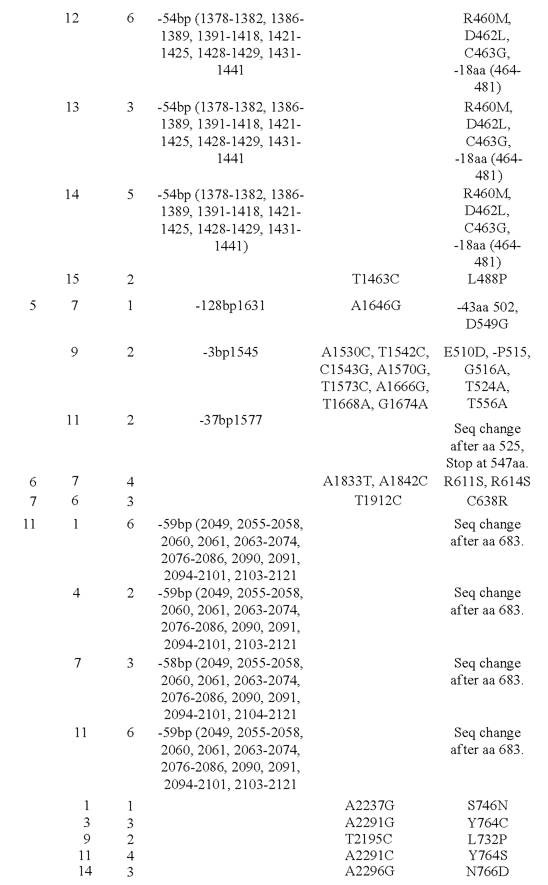

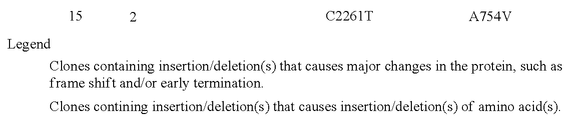

TABLE 3A
Human Subjects 1-88 and 91-99 (cDNA data) - List of GR Exon 2-3 Segment
Clones and Mutations

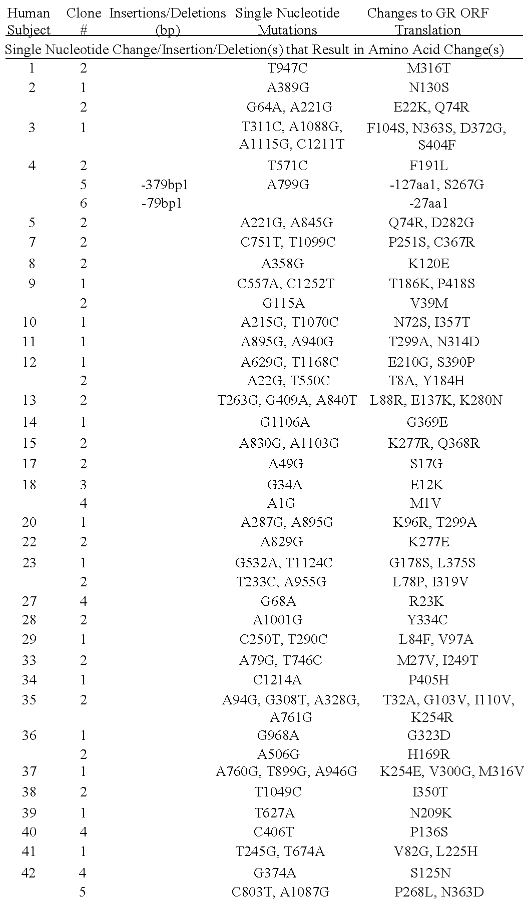

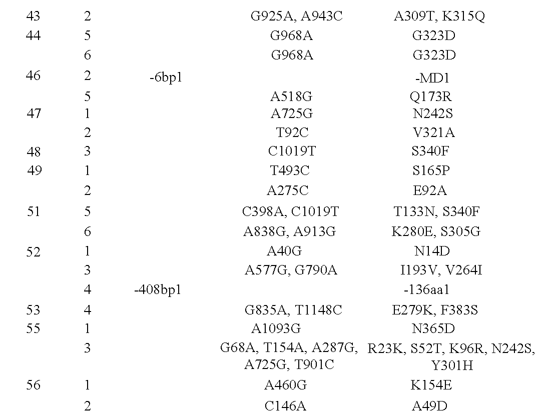

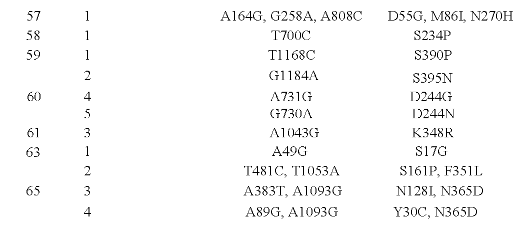

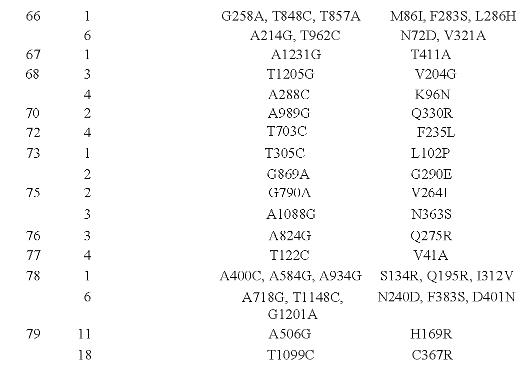

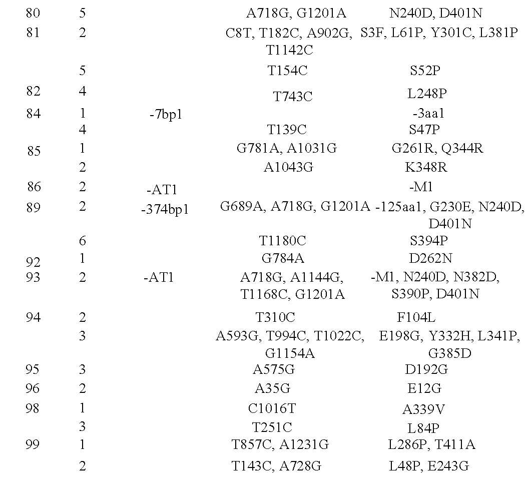

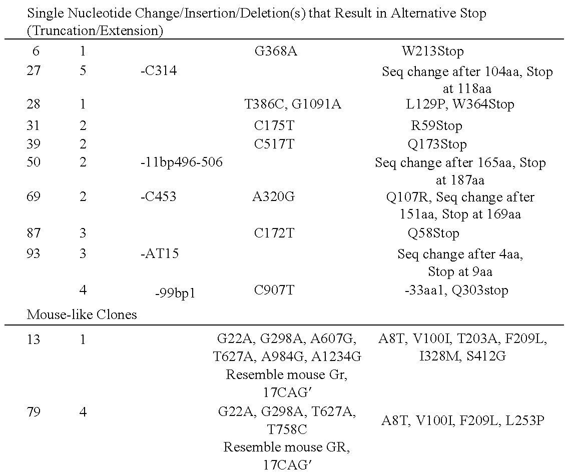

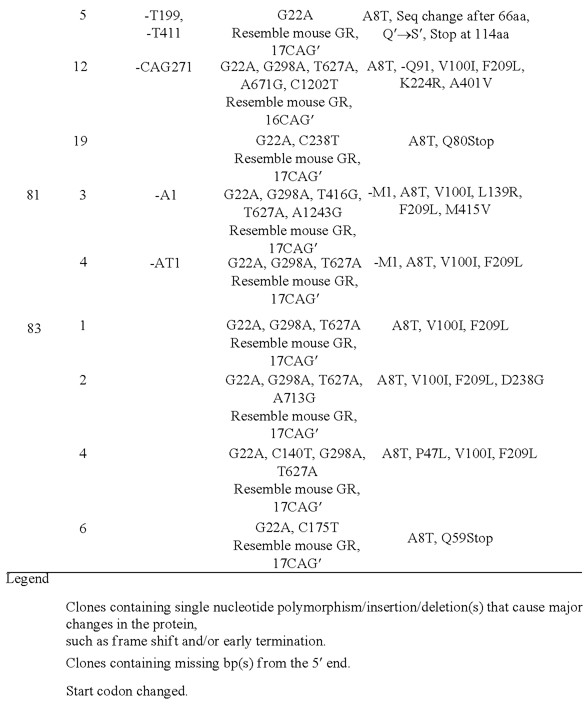

TABLE 3B
Human Subjects 1-88 and 91-99 (cDNA data) - List of Exon 3-9α Segment
Clones and Mutations

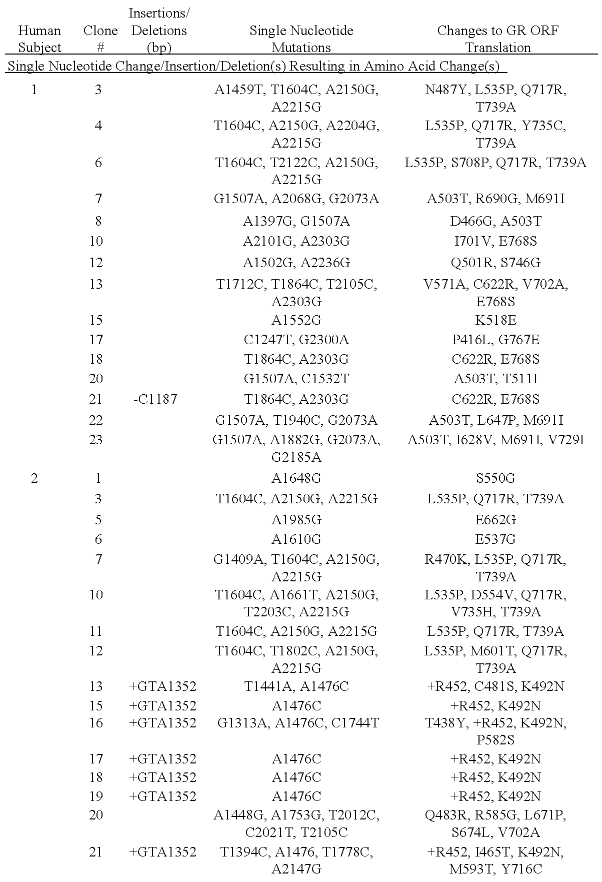

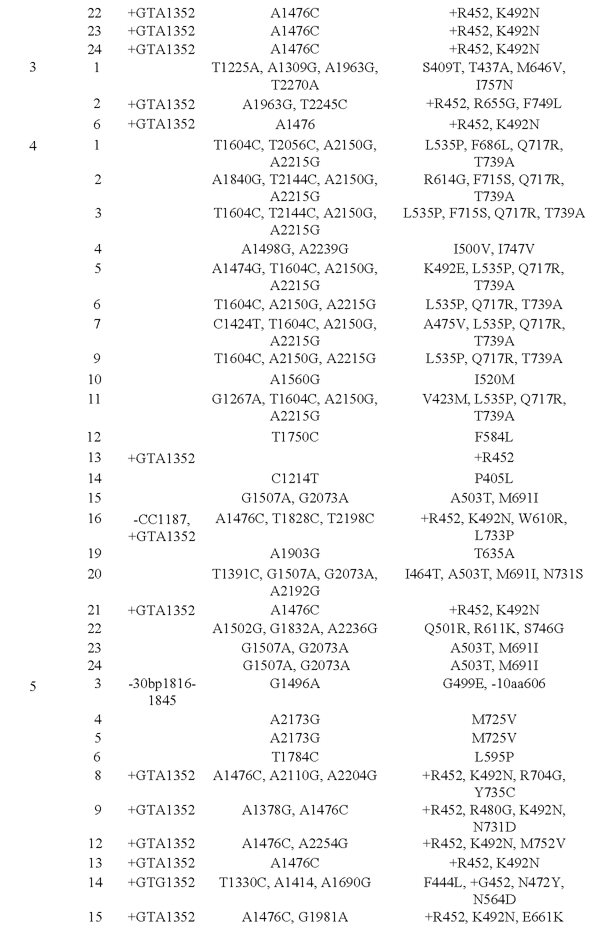

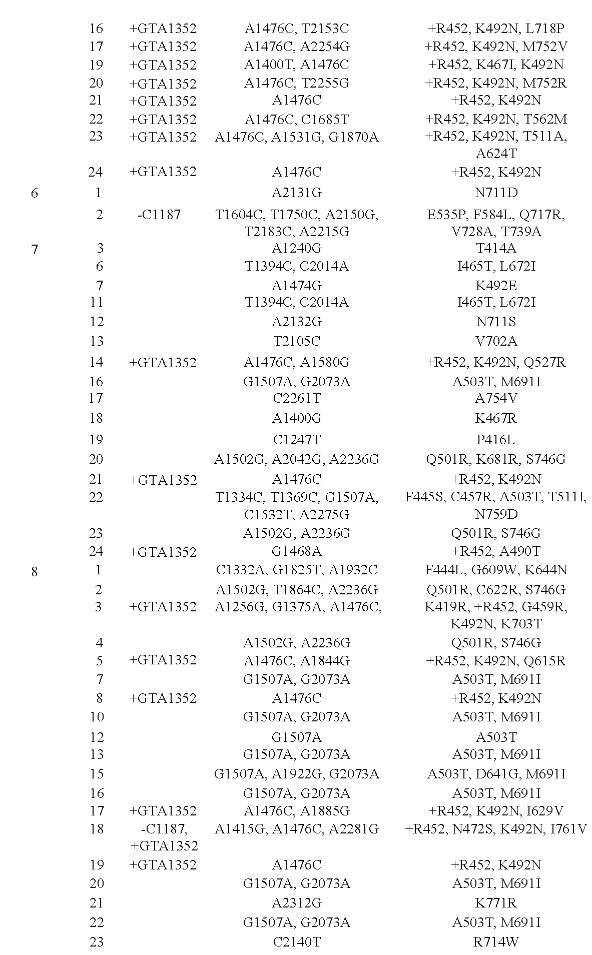

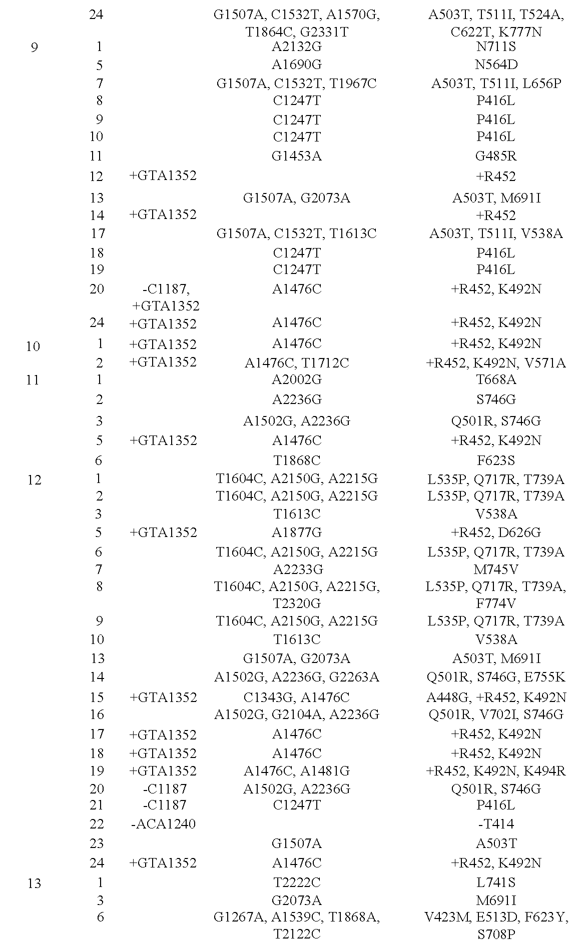

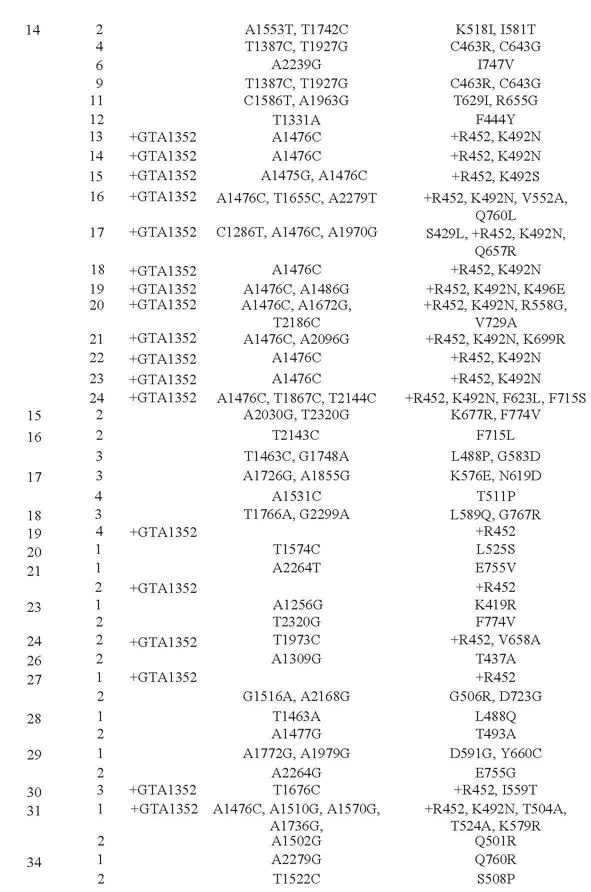

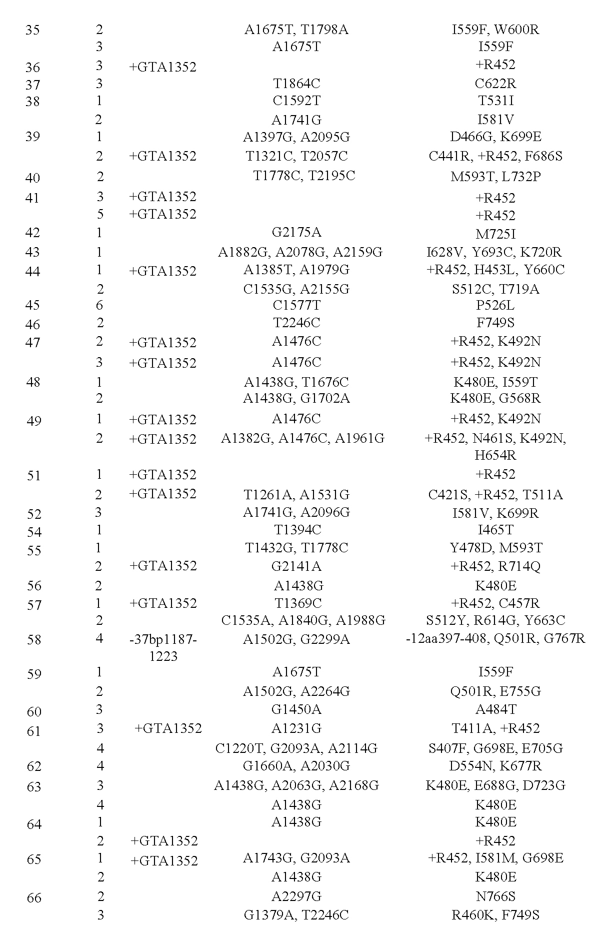

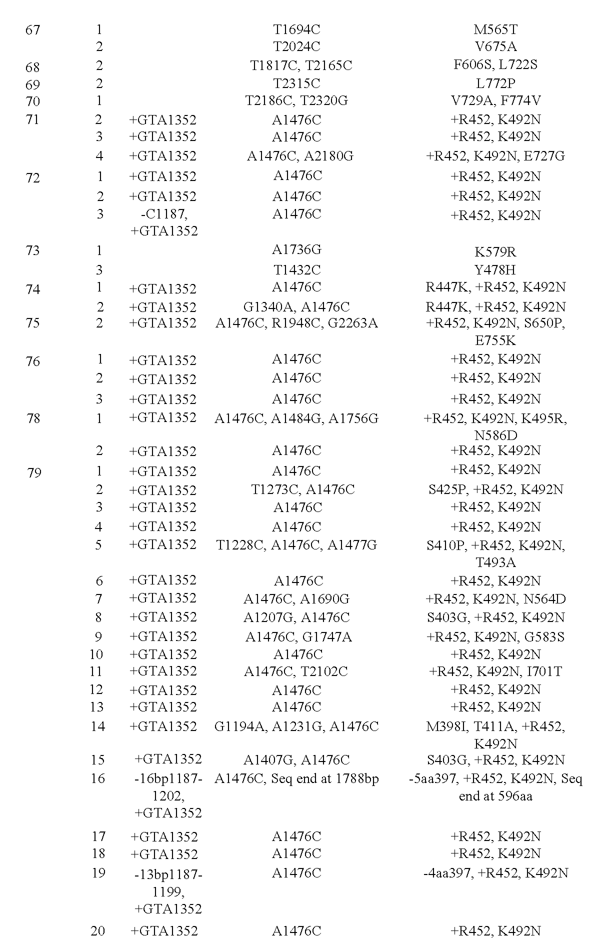

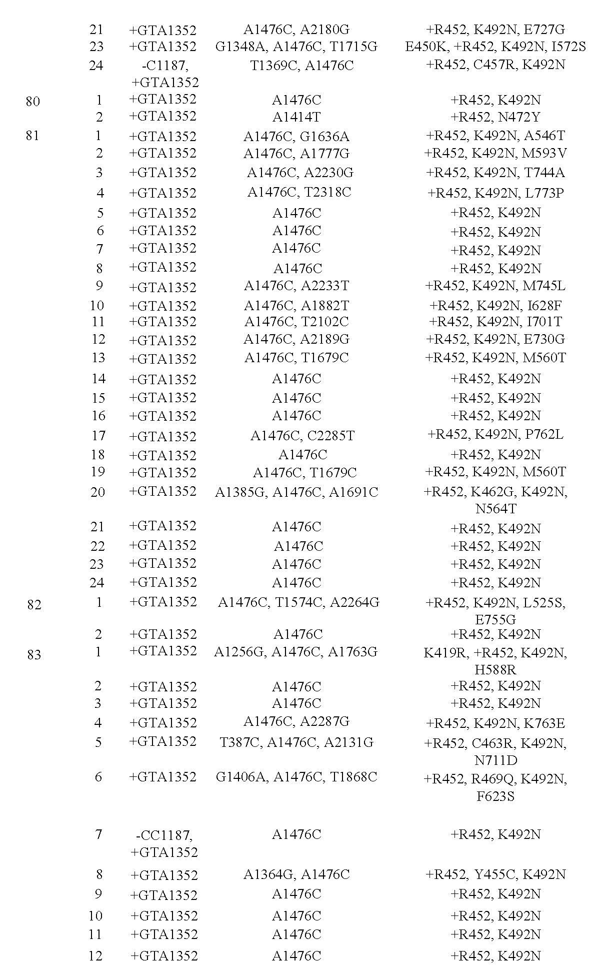

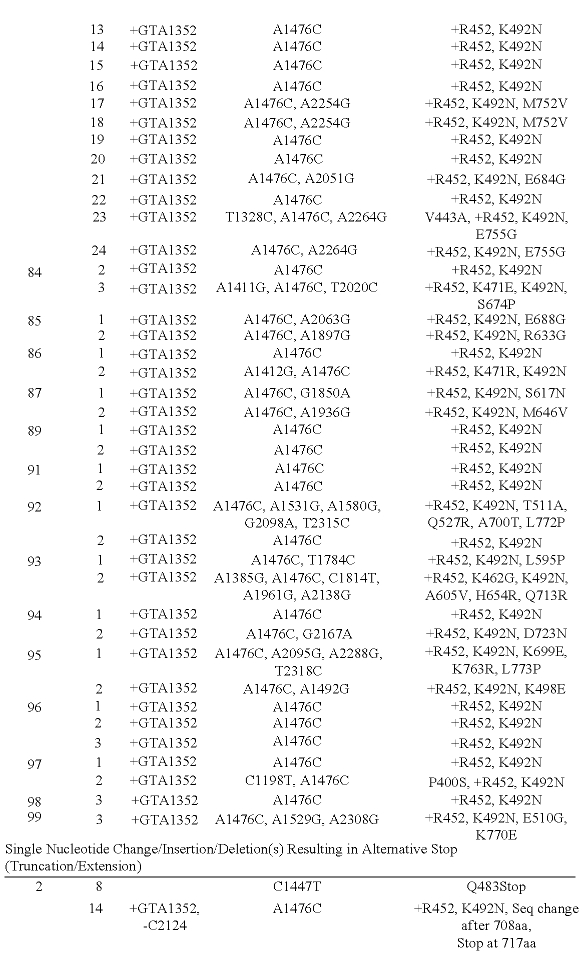

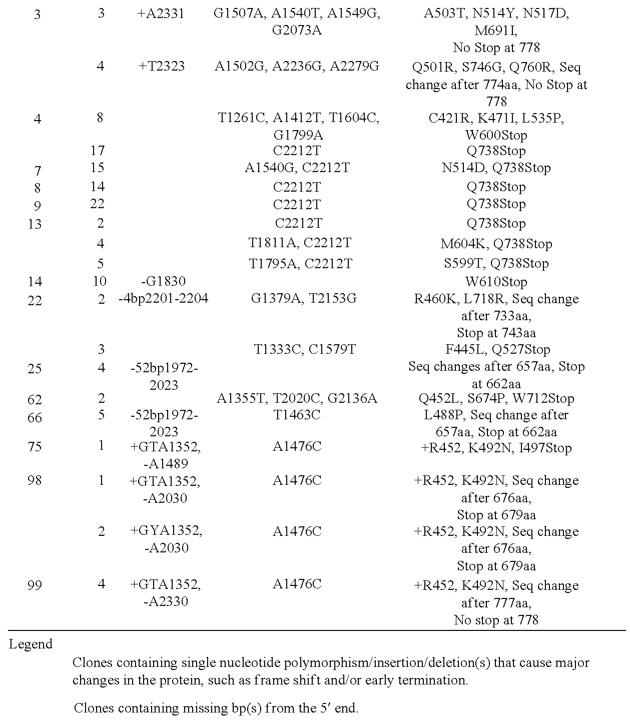

TABLE 4
Exon 2 Analysis of Subjects 79, 81, 83 (genomic DNA data) - List of Clones and
Mutations

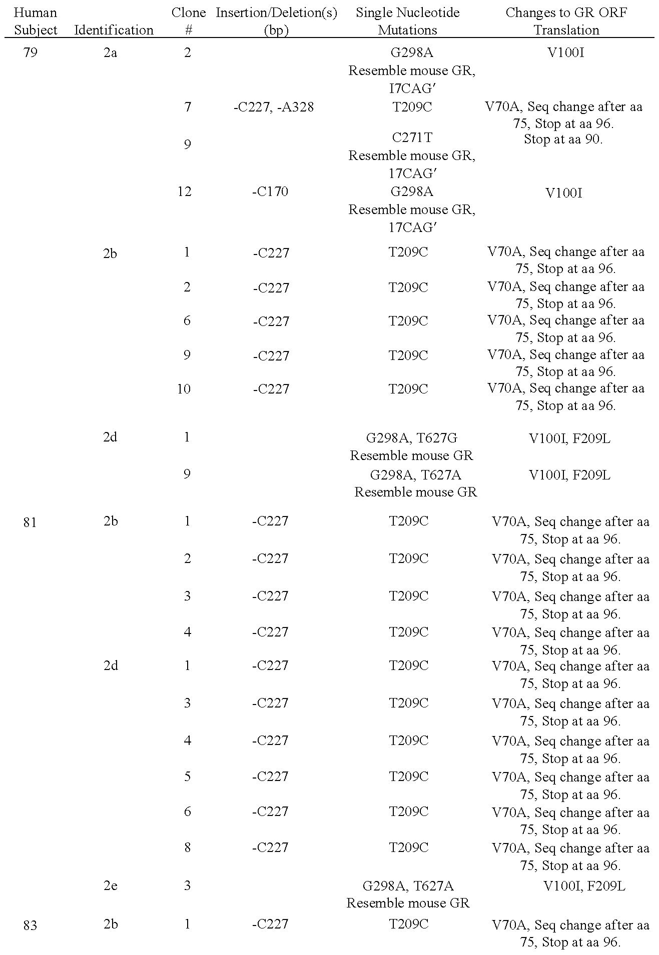

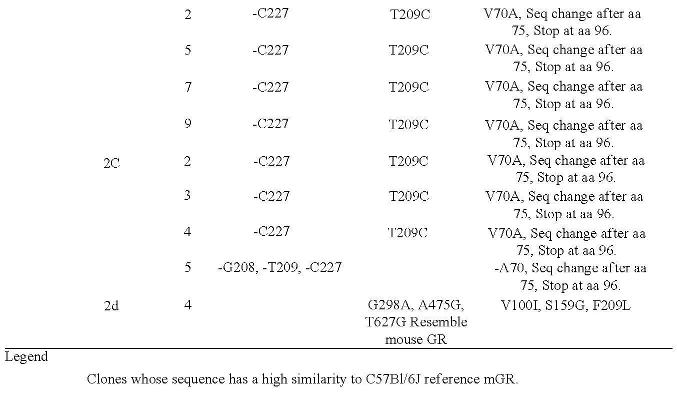

TABLE 5
Structure of Reconstructed human GR Alleles

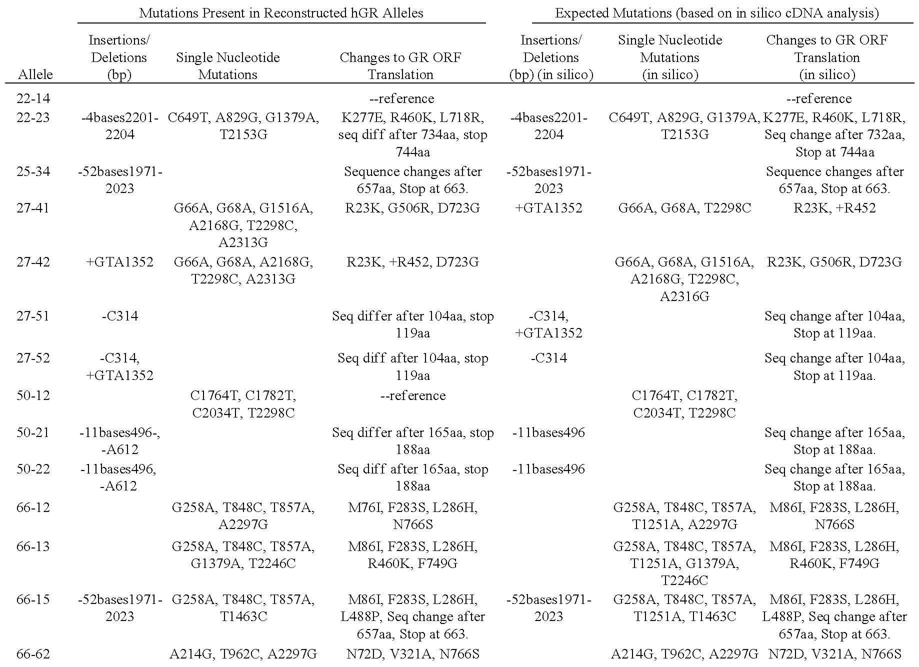

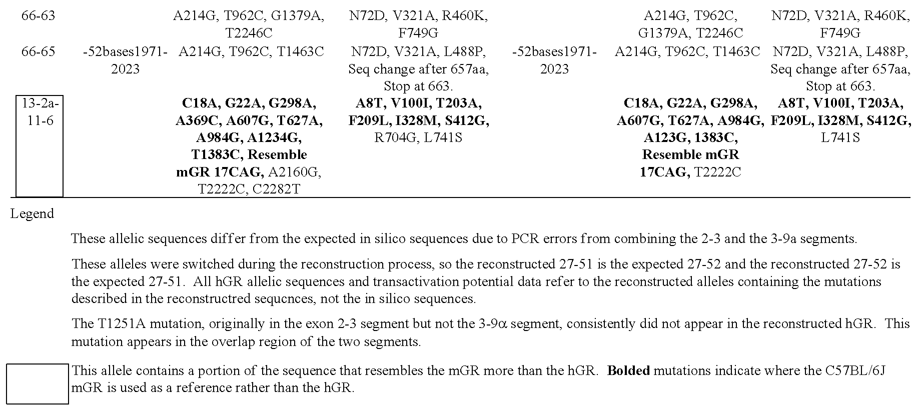

What is claimed is:

1. A method of determining whether a subject is resistant to glucocorticoid treatment, the method comprising:
obtaining a biological sample from the subject; and
analyzing the sample for the presence a glucocorticoid receptor (GR) nucleic acid comprising one or more mutations and encoding a GR polypeptide with decreased transactivation potential as compared to a reference GR polypeptide comprising SEQ ID NO: 2, and wherein the presence of the GR nucleic acid indicates that the subject is resistant to glucocorticoid treatment.

2. The method of claim 1, wherein the GR nucleic acid encodes a truncated GR polypeptide that lacks at least a portion of the ligand binding domain of the GR polypeptide.

3. The method of claim 1, wherein the GR nucleic acid comprises a G1379A mutation.

4. The method of claim 1, wherein the GR nucleic acid comprises a T2246C mutation.

5. The method of claim 1, wherein the GR polypeptide comprises a R460K amino acid substitution.

6. The method of claim 1, wherein the GR polypeptide comprises a F749G amino acid substitution.

7. The method of claim 1, wherein the GR nucleic acid comprises 17 CAG repeats in exon 2.

8. The method of claim 1, wherein the GR polypeptide comprises 17 glutamine repeats in the transactivation domain of the GR polypeptide.

9. The method of claim 1, wherein analyzing comprises isolating genomic DNA from the sample, performing polymerase chain reaction (PCR) on the genomic DNA using at least one primer selected from the group consisting of SEQ ID NOs: 3-61, and sequencing the PCR product.

10. The method of claim 1, wherein analyzing comprises detecting the GR polypeptide using an antibody that binds specifically to the GR polypeptide.

11. The method of claim 1, further comprising administer an agent or a treatment other than glucocorticoid to the subject if the presence of the GR nucleic acid with one or more mutations is detected.

12. A method of determining whether a subject is hypersensitive to glucocorticoid treatment, the method comprising:
obtaining a biological sample from the subject; and
analyzing the sample for the presence a glucocorticoid receptor (GR) nucleic acid comprising one or more mutations and encoding a GR polypeptide with increased transactivation potential as compared to a reference GR polypeptide comprising SEQ ID NO: 2, and wherein the presence of the GR nucleic acid indicates that the subject is hypersensitive to glucocorticoid treatment.

13. The method of claim 12, wherein the GR nucleic acid comprises a A2297G mutation.

14. The method of claim 12, wherein the GR polypeptide comprises a N766S amino acid substitution.

15. The method of claim 12, wherein analyzing comprises isolating genomic DNA from the sample, performing polymerase chain reaction (PCR) on the genomic DNA using at least one primer selected from the group consisting of SEQ ID NOs: 3-61, and sequencing the PCR product.

16. The method of claim 12, wherein analyzing comprises detecting the GR polypeptide using an antibody that binds specifically to the GR polypeptide.

17. The method of claim 12, further comprising administering a low dosage of glucocorticoid to the subject if the presence of the GR nucleic acid with one or more mutations is detected.

18. A kit, the kit comprising:
(a) means for detecting whether a subject has a GR nucleic acid sequence comprising one or more mutations; and
(b) instructional material for using the kit.
